(19) United States
(12) Patent Application Publication
(10) Pub. No.: US 2023/0211157 A1
(43) Pub. Date: Jul. 6, 2023

(54) NONINVASIVE CRANIAL NERVE THERAPY

(71) Applicant: MUSC Foundation for Research Development, Charleston, SC (US)

(72) Inventors: Bashar Badran, San Ramon, CA (US); Mark George, Sullivans Island, SC (US); Doe Jenkins, Johns Island, SC (US); Daniel Cook, Charleston, SC (US)

(21) Appl. No.: 18/000,011

(22) PCT Filed: May 28, 2021
(86) PCT No.: PCT/US2021/034918

§ 371 Date: Nov. 28, 2022

Related U.S. Application Data
(60) Provisional application No. 63/031,522, filed on May 28, 2020.

(51) Int. Cl. A61N 1/36; A61N 1/04

(52) U.S. Cl. CPC A61N 1/36003; A61N 1/0456; A61N 1/0472; A61N 1/36031; A61N 1/36034

(57) ABSTRACT

The present invention relates to systems for providing noninvasive cranial nerve stimulation and methods for using the same. The present invention administers therapy through electrodes that are noninvasively attached to one or more of a subject’s cranial nerve. The systems can be used to enhancing rehabilitation and recovery by improving neuroplasticity and coupling muscle training with feedback.

### CROSS-REFERENCE TO RELATED APPLICATIONS

This application claims priority to U.S. Provisional Pat. Application No. 63/031,522, filed May 28, 2020, the contents of which are incorporated by reference herein in its entirety.

### STATEMENT REGARDING FEDERALLY SPONSORED RESEARCH OR

### DEVELOPMENT

This invention was made with government support under Grant No. P2HCD086844 awarded by the National Institute of Health. The government has certain rights in the invention.

### BACKGROUND OF THE INVENTION

Preterm infants and term infants who suffer hypoxic ischemic encephalopathy (HIE) are at high risk for motor problems, which primarily manifest as feeding delays during their neonatal hospital admission. Oromotor dyscoordination is very common in both groups of infants, and typically takes 3-6 weeks of working on oral feedings in the hospital before the infant may take enough breast milk or formula to sustain adequate growth for discharge. Occupational therapy usually works with infants once a day to ensure that the feeding particulars, such as nipple choice, frequency of oral feeding, do not tax infant physiology too greatly and to guide learning this motor skill. Feeding difficulty is the primary reason for delayed discharge of preterm or HIE infants. Many of these infants will not be able to master this motor skill before term age (40-42 weeks gestation) and will receive a gastrostomy tube (G-tube) for direct gastric feeding, in order that they may finally be discharged from the hospital to home. Neonatal intensive care units (NICU) place on average 40 G-tubes per year. This procedure requires general anesthesia for both insertion and eventual take down, and leaves scars in the epigastric area. The g-tube also reinforces the parental perception that their child is not normal and that he or she has a more limited developmental potential than a ‘normal’ child.

Even with significant brain injury, it is known that neuroplasticity in infants may lead to improved, and even near normal outcomes. This neuroplasticity involves stimulating neurogenesis and reparative inter-neuronal connections to improve motor skills in neonatal animal models and in adults after stroke. In addition, it is known that rehabilitative training may be enhanced by brain stimulation using a variety of modalities.

Feeding in neonates involves a sequence of sucking, swallowing, and breathing that requires coordination of the face, head, and neck muscles with the myelinated vagal regulation of the bronchi and the heart. In preterm neonates, the muscles needed to feed are underdeveloped, resulting in the need for OT rehabilitation to ‘learn’ feeding patterns. Preterm neonates’ inability to feed effectively is the primary reason for prolonged hospital stays. In neonates with HIE, development of cortex and basal ganglia is interrupted, and depending on the severity, normal developmental plasticity is hindered, further contributing to their inability to feed. Both types of feeding difficulties involve complex motor learning, which requires integration of sensory and motor pathways.

Thus, there is a need in the art for improved systems and methods for administering neural stimulation for enhancing neuroplasticity and muscle training. The present invention meets this need.

### SUMMARY OF THE INVENTION

In one aspect, the present invention provides a method of enhancing oromotor skills, comprising the steps of: providing a cranial nerve stimulation system comprising at least one means of triggering activation and at least one stimulating electrode; securing the at least one stimulating electrode adjacent to a subject’s cranial nerve; providing the subject with a source of food; initiating feeding upon a trigger of activation; and administering stimulation using the at least one stimulating electrode to the cranial nerve in response to the trigger of activation.

In one embodiment, the at least one means of triggering activation is selected from the group consisting of: a sensing electrode noninvasively secured adjacent to a subject’s cheek or jaw muscle, a sensing mechanism integrated into a nipple or bottle used in feeding, or a manual switch. In one embodiment, the trigger of activation is selected from the group consisting of: a sensing electrode sensing muscle activation that surpasses a minimum threshold, a sensing electrode sensing some displacement of food, or a manual switch being activated.

In one embodiment, the cranial nerve is selected from the group consisting of: the trigeminal nerve, the facial nerve, the accessory nerve, the hypoglossal nerve, the auricular branch of the vagus nerve, and the main bundle of the vagus nerve. In one embodiment, the measuring step and the administering step are repeated in a closed loop. In one embodiment, the administering step is performed manually in an open loop. In one embodiment, the at least one stimulating electrode is noninvasively secured to a subject’s ear canal, tragus, cymba conchae, lobe, helix, anti-helix, mastoid, or neck.

In one embodiment, the minimum threshold is an absolute value selected from the group consisting of about: 0.1 µV 0.5 µV 1 µV 5 µV 10 µV 50 µV 100 µV 200 µV 300 µV 400 µV 500 µV 1 mV, 5 mV, 10 mV, 20 mV, 30 mV, 40 mV, or 50 mV. In one embodiment, the minimum threshold is a change from a base measurement taken at rest selected from the group consisting of about: 0.1 µV 0.5 µV 1 µV 5 µV 10 µV 50 µV 100 µV 200 µV 300 µV 400 µV 500 µV 1 mV, 5 mV, 10 mV, 20 mV, 30 mV, 40 mV, or 50 mV. In one embodiment, the minimum threshold is a percentage of a maximum potential of the muscle selected from the group consisting of about: 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95%.

In one embodiment, the stimulation has an intensity selected from the group consisting of about: 0.01 mA, 0.05 mA, 0.1 mA, 0.2 mA, 0.3 mA, 0.4 mA, 0.5 mA, 0.6 mA, 0.7 mA, 0.8 mA, 0.9 mA, 1 mA, 1.5 mA, 2 mA, 2.5 mA, 3 mA, 3.5 mA, 4 mA. 4.5 mA, 5 mA. 6 mA, 7 mA, 8 mA, 9 mA, and 10 mA. In one embodiment, the stimulation has a frequency selected from the group consisting of about: 1 Hz, 2 Hz, 3 Hz, 4 Hz, 5 Hz, 6 Hz, 7 Hz, 8 Hz, 9 Hz, 10 Hz, 15 Hz, 20 Hz, 25 Hz, 30 Hz, 35 Hz, 40 Hz, 45 Hz, and 50 Hz. In one embodiment, the stimulation has a pulse width selected from the group consisting of about: 10 µs, 20 µs, 30 µs, 40 µs, 50 µs, 60 µs, 70 µs, 80 µs, 90 µs, 100 µs, 150 µs, 200 µs, 250 µs, 300 µs, 350 µs, 400 µs, 450 µs, 500 µs, 550 µs, 600 µs, 650 µs, 700 µs, 750 µs, 800 µs, 850 µs, 900 µs, 950 µs, and 1 ms. In one embodiment, the stimulation has an on duration and an off duration, each selected from the group consisting of about: 0.1 seconds, 0.5 seconds, 1.5 seconds, 2 seconds, 2.5 seconds, 3 seconds, 3.5 seconds, 4 seconds, 4.5 seconds, 5 seconds, 10 seconds, 20 seconds, 30 seconds, 40 seconds, 50 seconds, 1 minute, 2 minutes, 3 minutes, 4 minutes, 5 minutes, 10 minutes, 15 minutes, 20 minutes, 30 minutes, 40 minutes, 45 minutes, 50 minutes, and 1 hour.

In one embodiment, the method increases white matter neuroplasticity as measured by increased tissue anisotropy. In one embodiment, tissue anisotropy is increased in white matter tracts selected from the group consisting of: the anterior limb of internal capsule, inferior front-occipital fasciculus, external capsule, and superior longitudinal fasciculus.

In one aspect, the present invention provides a cranial nerve stimulation system, comprising: at least one stimulating electrode configured to attach adjacent to a cranial nerve; at least one switch electrically connected to the at least one stimulating electrode; and at least one stimulating unit electronically connected to the at least one stimulating electrode and the at least one switch; wherein the at least one switch is configured to activate the at least one stimulating electrode to stimulate the cranial nerve, and wherein the at least one stimulating unit is configured to modulate at least one stimulation parameter.

In one embodiment, the at least one cranial nerve is selected from the group consisting of: the trigeminal nerve, the facial nerve, the accessory nerve, the hypoglossal nerve, the auricular branch of the vagus nerve, and the main bundle of the vagus nerve.

In one embodiment, the system further comprises an impedance sensor indicating to the user whether the at least one stimulating unit has good, fair or poor contact with the patient.

In one embodiment, the system further comprises a sensing electrode configured to attach adjacent to at least one muscle, power source, a transmitter, and a processor communicatively connected to a non-transitory computer-readable memory with instructions store thereon, which when executed by the processor, initiates a closed-loop synchronization between activation and deactivation of the at least one stimulating electrode when the at least one sensing electrode measures electrical energy in the at least one muscle that passes a minimum threshold.

In one embodiment, the system further comprises a feeding bottle comprising at least one sensor, a power source, and a transmitter. In one embodiment, the at least one sensor is selected from the group consisting of: a flow sensor, a pressure sensor, a suction sensor, a gyroscope, an accelerometer, a temperature sensor, and a volume sensor.

In one embodiment, the stimulating electrode comprises an earpiece and a conductive element. In one embodiment, the earpiece comprises at least one outer surface conductive region positioned to directly contact a region of an ear. In one embodiment, the earpiece is an anode and the conductive element is a cathode.

In one aspect, the present invention provides a method of enhancing muscle rehabilitation, comprising the steps of: providing a cranial nerve stimulation system at least one stimulating electrode, at least one switch electrically connected to the at least one stimulating electrode, and at least one stimulating unit electronically connected to the at least one stimulating electrode and the at least one switch; securing the at least one stimulating electrode to a subject’s cranial nerve; setting at least one stimulating parameter using the at least one stimulating electrode; and administering stimulation using the at least one stimulating electrode to the cranial nerve.

In one embodiment, the cranial nerve is selected from the group consisting of: the trigeminal nerve, the facial nerve, the accessory nerve, the hypoglossal nerve, the auricular branch of the vagus nerve, and the main bundle of the vagus nerve. In one embodiment, the administering step is performed manually in an open loop by activating the at least one switch.

In one embodiment, the present invention provides a method indicating to the user whether the at least one stimulating unit has good, fair or poor contact with the patient.

In one embodiment, the system further comprises at least one sensing electrode and the method further comprises a step of securing the at least one sensing electrode adjacent to a subject’s muscle group of interest, a step of measuring muscle group activation using the at least one sensing electrode that surpasses a minimum threshold, and the measuring step and the administering step are repeated in a closed loop in response to the measurement of muscle group activation surpassing the minimum threshold.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1A and FIG. 1B depict diagrams showing exemplary systems for pairing noninvasive cranial nerve stimulation with neonate feeding via a smart bottle. FIG. 1C and FIG. 1D depicts a diagram showing an exemplary system for pairing noninvasive cranial nerve stimulation with neonate feeding via a manual trigger switch (wired and wireless activation). FIG. 1D depicts an exemplary stimulating unit. FIG. 1F depicts an exemplary wearable device for noninvasive cranial nerve stimulation.

FIG. 2 depicts a diagram showing an exemplary system for triggering cranial nerve stimulation in neonate feeding.

FIG. 3 depicts a diagram showing an exemplary system for manually triggering cranial nerve stimulation in neonate feeding.

FIG. 4 depicts a diagram showing an exemplary system for triggering cranial nerve stimulation in muscle rehabilitation.

FIG. 5 depicts a flowchart for an exemplary method of training neonate feeding.

FIG. 6 depicts a flowchart for an exemplary method of training muscle rehabilitation.

FIG. 7 depicts exemplary electromyography electrode placement for muscle activation detection and stimulation in training neonate feeding behavior when utilizing a sensing electrode to measure muscle activation as the means of activating stimulation.

FIG. 8 depicts the results of experiments investigating optimal electrode placement that delivers the most reliable stimulation trigger induced by a visual suck in neonate feeding.

FIG. 9 depicts the results of experiments investigating optimal electrode placement that delivers the highest rate of stimulation when a visual suck is recorded in neonate feeding.

FIG. 10 depicts historical feeding data in a sample of infants having feeding difficulty.

FIG. 11A depicts the results of administering cranial nerve therapy to 14 babies having feeding difficulty. FIG. 11B demonstrates the comparison of administering cranial nerve therapy once daily versus twice daily.

FIG. 12 depicts the results of statistical analysis for the 8 responders in the treatment group shown in FIG. 11A; the responders have significant changes in their oral feeding behavior, indicated by significant changes in the slopes of their linear regression lines.

FIG. 13 depicts the results of statistical analysis for the 6 non-responders in the treatment group shown in FIG. 10; the non-responders have linear regression slopes that are non-significantly different from zero, indicating that no improvement has been achieved.

FIG. 14A and FIG. 14B depict the results of experiments investigating the effect of cranial nerve therapy on brain white matter tract integrity in infants. FIG. 14A shows fractional anisotropy (FA) change per week between responders (full feed) and non-responders (G-tube) in the Left External Capsule and Right Corpus Callosum, two white matter regions of interest important in motor integration. FIG. 14B shows axial kurtosis (K_{||}) change per week between responders (full feed) and non-responders (G-tube) in Left Posterior Thalamic Radiations (PTR) and Right Inferior Front-Occipital Fasciculus (IFOF), two white matter regions of interest important in sensorimotor integration.

FIG. 15 depicts an experimental overview of the study for assessment of the feasibility of combining taVNS with bottle feeding.

FIG. 16 depicts the result of experiments investigating achievement of full oral feeds between Responders with taVNS treatment versus Non-Responders who received G-tube.

FIG. 17 depicts the result of experiments investigating dose response between 1X Responders and 2X Responders.

FIG. 18 depicts the result of experiments investigating Responders versus Non-Responders in corpus callosum and external capsule, per week of development.

FIG. 19 depicts the result of experiments investigating G-tube feeds in the corpus callosum in both right and left hemispheres.

FIG. 20 depicts improvement in head and neck control and overall functional movements. The regions highlighted in red are significantly higher in FA in the infants who performed well with Rolling by Arm, corresponding to left sided portions of major WM tracts: the anterior limb of internal capsule (ALIC) and inferior fronto-occipital fasciculus (IFOF) in the first row of images; the external capsule (EC) with ALIC in the second row; and the superior longitudinal fasciculus (SLF) in third row. This demonstrates that tissue anisotropy is increasing more rapidly in high performing infants than their low performing counterparts, potentially showing that increased WM maturation in specific WM tracts is likely reflecting neuroplasticity induced by taVNS-paired bottle feeding.

### DETAILED DESCRIPTION

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Cranial Nerve Stimulation Systems

The present invention is based in part on systems for providing noninvasive cranial nerve stimulation. The systems administer therapy through electrodes that are noninvasively attached to one or more of a subject’s cranial nerve. The systems can be used to enhancing rehabilitation and recovery by improving neuroplasticity and coupling muscle and reflex training with feedback.

Stimulation can be noninvasively administered to any suitable cranial nerve. Non-limiting examples include the trigeminal nerve, the facial nerve, the accessory nerve, the hypoglossal nerve, the auricular branch of the vagus nerve, the main bundle of the vagus nerve, and the like. The auricular branch of the vagus nerve can be accessed in a variety of ways, including but not limited to the ear canal, the tragus, the cymba conchae, the outer ear, the mastoid, and combinations thereof. In various embodiments, any portion of the ear can be used for nerve stimulation. In other embodiments, the area of the face and head directly surrounding the ear can be used for nerve stimulation. The main bundle of the vagus nerve can be accessed at any suitable location along the neck. In various embodiments, the stimulation is administered transcutaneously. Stimulation can be administered using one or more electrodes secured adjacent to a cranial nerve in any suitable manner, including but not limited to using an adhesive, a clip, a patch, an ear plug, a head band, a neck brace, a collar, a head covering, and the like.

In some embodiments, the present invention provides therapeutic tools aimed at improving and accelerating learned feeding behavior in neonates. The systems provided change the way rehabilitation is conducted for preterm neonates, resulting in earlier discharge, lower hospital costs, improved parental perception of the developmental potential of their infant, and reduces stress and improves bonding with parents, both in and out of the hospital. The systems can help neonates avoid gastrointestinal tube feeding by training feeding behavior such that neonates are able to achieve full oral feeds (about 120 mL/kg/d with weight gain). The systems can serve as a take-home feeding aid for convalescing critically ill infants who have missed the developmental window to master the feeding skill, and for infants with congenital syndromes that make oral feeding challenging.

Treating oromotor difficulties during the learned task of feeding with noninvasive brain stimulation that promotes plasticity, poses a highly novel application of transcutaneous auricular vagus nerve stimulation (taVNS). The major premise is that in babies at high risk for motor problems, simultaneously delivered brain stimulation via taVNS will boost motor cortical plasticity involved in a learned feeding task, leading to better feeding. Previous efforts having required surgically implanted VNS and were not attempted in neonates. This invention utilizes novel forms of noninvasive vagus nerve stimulation (nVNS) (rather than surgically implanted) paired with feeding to accelerate and enhance the learning of feeding in neonates.

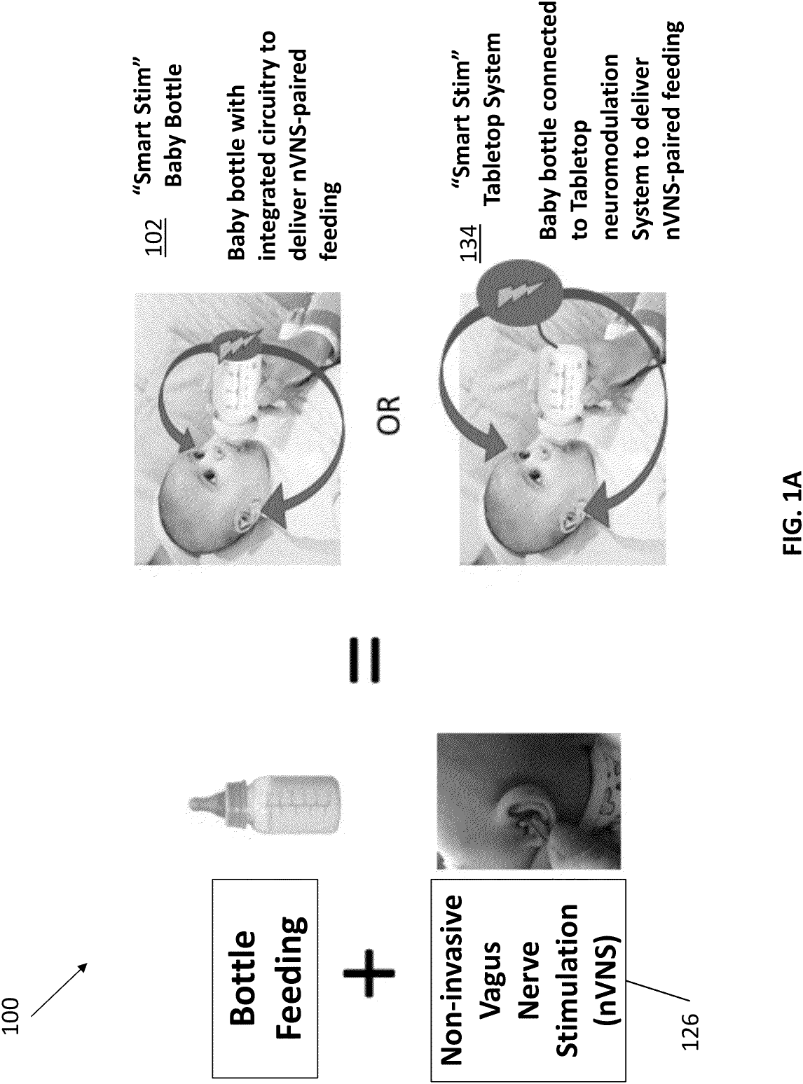

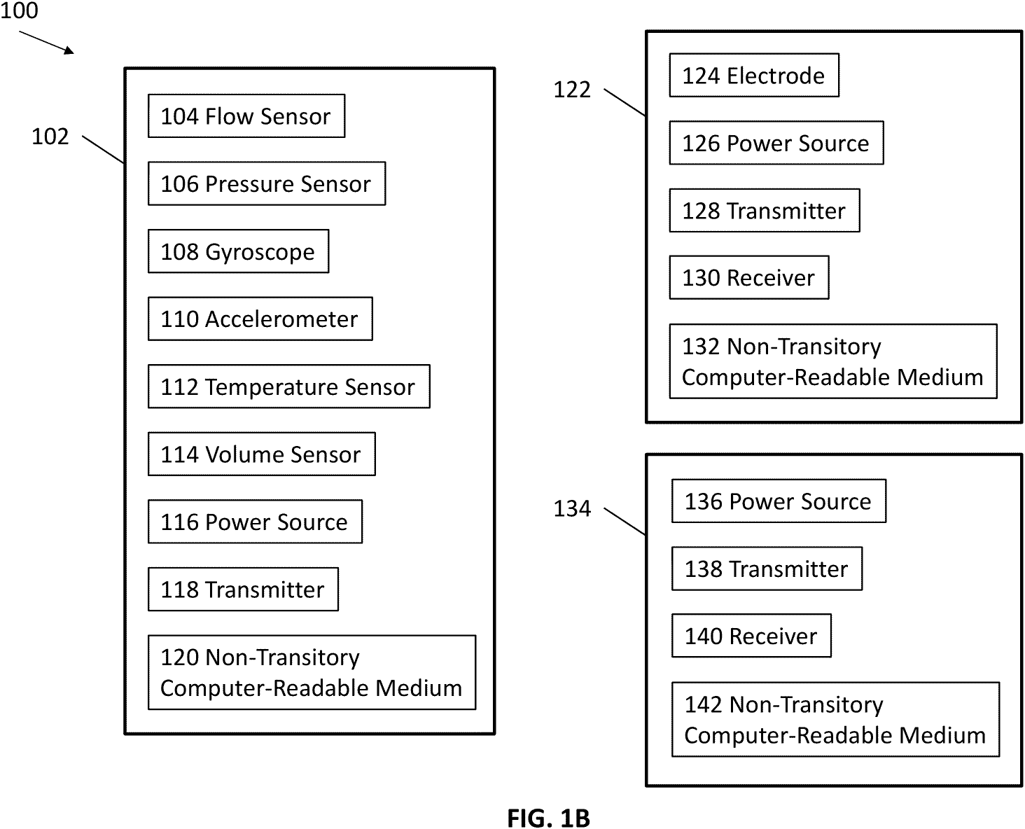

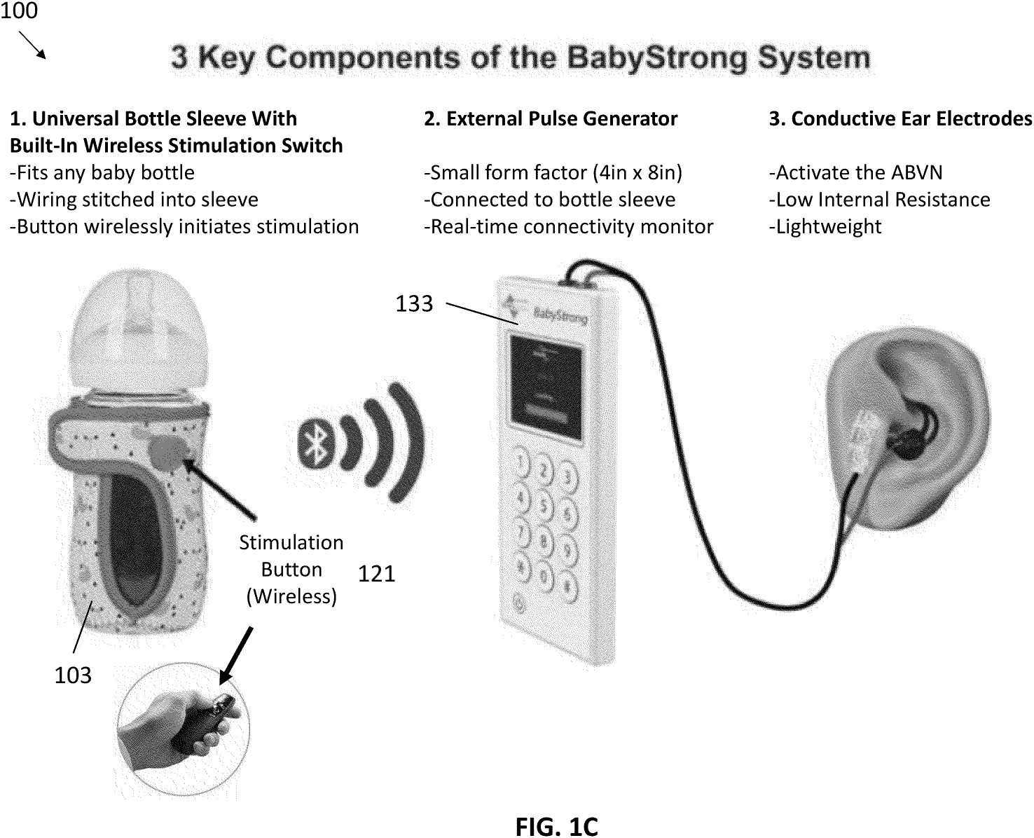

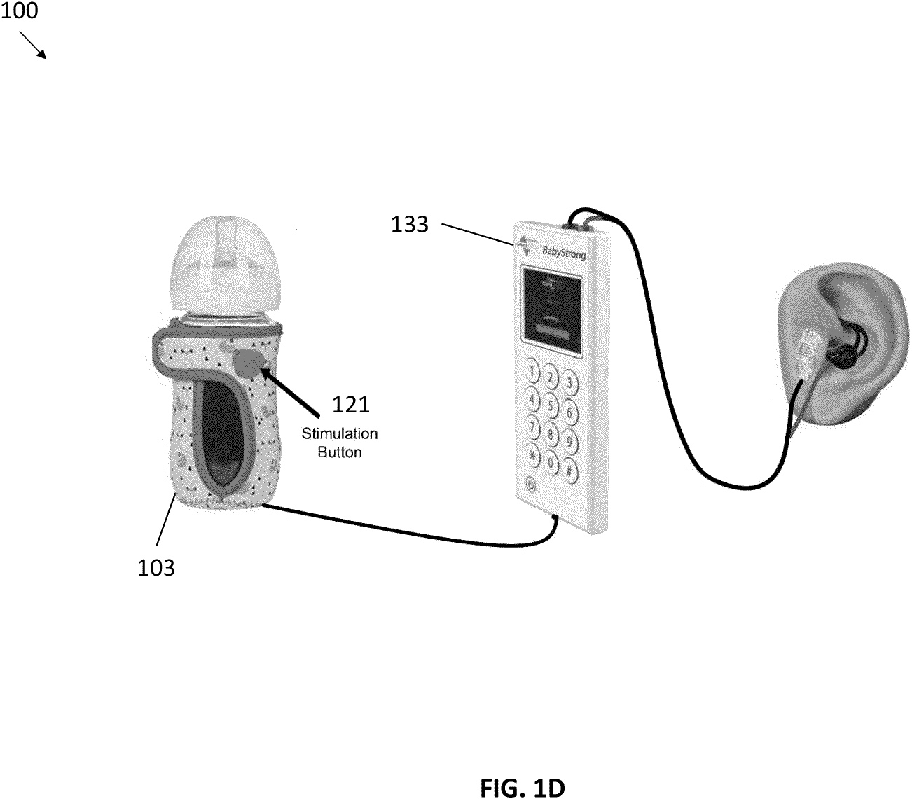

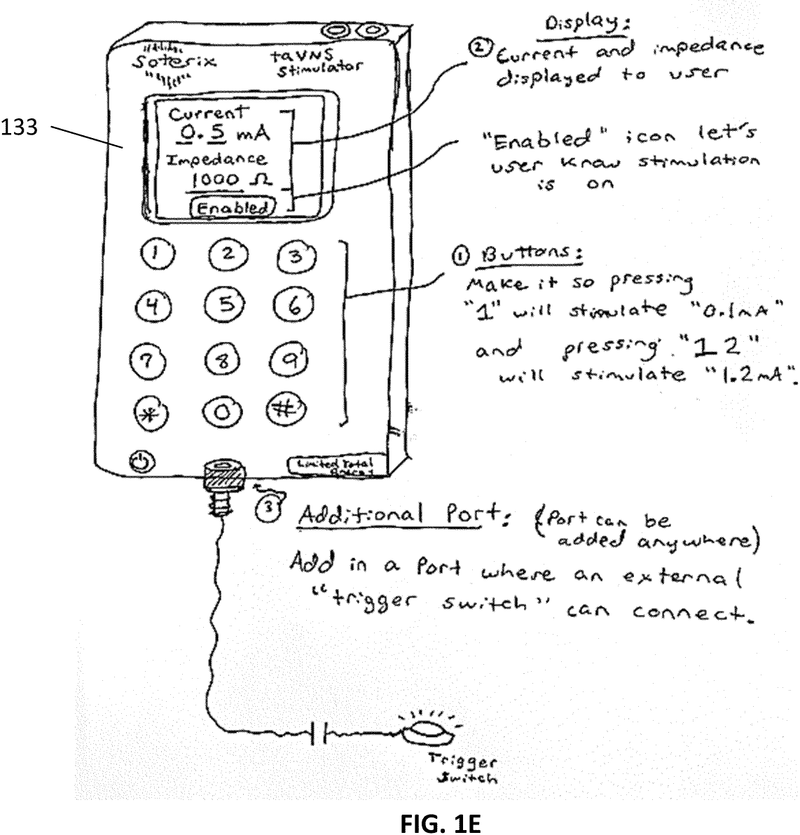

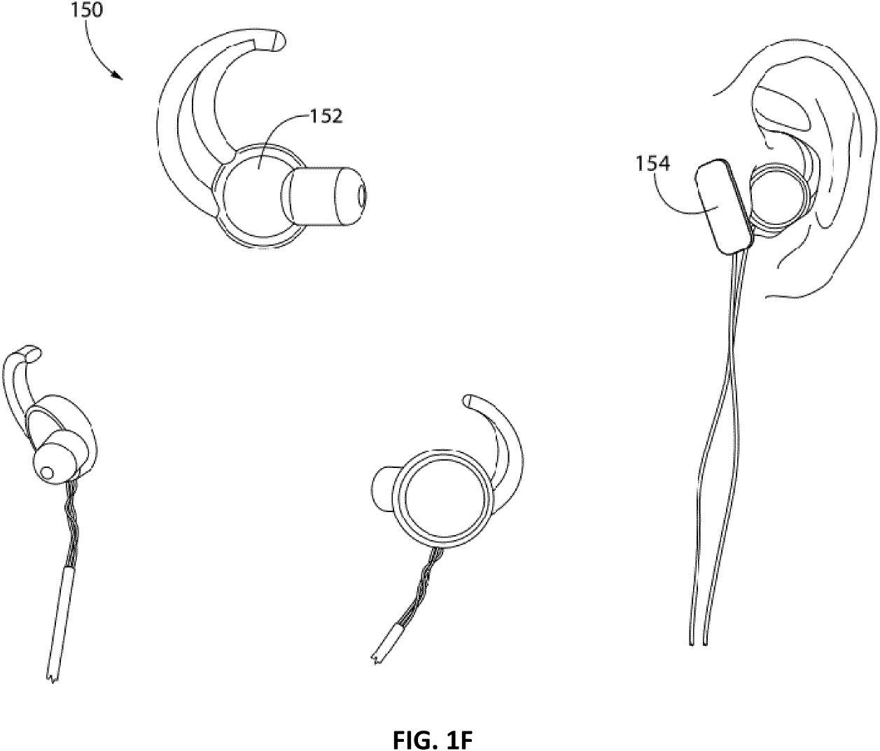

Referring now to FIG. 1A and FIG. 1B, an exemplary system 100 is depicted. In various embodiments, system 100 comprises several components that can be used alone or in combination to couple cranial nerve stimulation with feedback to train feeding behavior in infants. For example, in some embodiments system 100 comprises bottle 102, wearable 122, and computer platform 134.

Bottle 102 can comprise any desired feeding bottle with reservoir connected to a mouthpiece having a nipple or other aperture suitable for engaging an infant’s mouth typically used for feeding infants, with the further addition of at least one flow sensor 104, pressure sensor 106, gyroscope 108, accelerometer 110, temperature sensor 112, volume sensor 114, and combinations thereof. The at least one flow sensor 104 and pressure sensor 106 can be used to detect and measure the timing and amount of food obtained by an infant during a feeding session. The at least one gyroscope 108 and accelerometer 110 can be used to detect and measure the position of bottle 102 and monitor feeding behavior over time as a function of the movement of bottle 102. The at least one temperature sensor 112 can be used to monitor the temperature of bottle 102 to indicate whether the contents are at a suitable temperature, or whether the contents are too cold or too hot for consumption. The at least one volume sensor 114 can be used to detect and measure the amount of food remaining in bottle 102. Any suitable volume sensor 114 can be used, including float sensors, ultrasonic level sensors, laser level sensors, and the like. Additional sensors are also contemplated, such as suction sensors, blood pressure sensors, pulse oximetry sensors, glucose sensors, and the like. In some embodiments, bottle 102 can be powered by a power source 116 (such as a battery or an electrical plug). In some embodiments, bottle 102 can further include a wired or wireless transmitter 118 for transmitting data collected by the various sensors, and a non-transitory computer-readable medium 120 connected to a processor to store data collected by the various sensors. In some embodiments, bottle 102 comprises one or more switches for manual activation and deactivation of the abovementioned components.

Referring now to FIG. 1C and FIG. 1D, a variation of exemplary system 100 is depicted. In various embodiments, the components of system 100 can be modified to couple cranial nerve stimulation with feedback to train feeding behavior in infants. For example, in some embodiments the means of activating stimulation can be a user-activated trigger switch 121 integrated into a sleeve 103 or other covering that is securable to a bottle. In some cases, switch 121 is integrated into bottle 102 itself. In other embodiments, trigger switch 121 can be any actuatable wireless switch. Switch 121 can be located in any position that is ergonomically appropriate for an individual administering feeding to a subject. Switch 121 can also be located in any position convenient for an individual administering or observing feeding of a subject so long as it is within wired or wireless communication with stimulating unit 133. Switch 121 can come in any form capable of activating and deactivating stimulation when triggered by a user. In various embodiments, switch 121 can be activated remotely or over a network. Since activation of switch 121 is not dependent on sensor activation, switch 121 can thereby be activated from any location and for any duration for cranial nerve stimulation and enhancing feeding behavior. Switch 121 can also be activated when not feeding, as a tool to generally improve neuroplasticity in neonates.

In some embodiments, switch 121 is a dead man’s type switch that applies stimulation only when the switch is activated. In some embodiments, switch 121 is a trigger switch that when activated initiates stimulation for a defined period of time until shutting off once the defined period of time is reached or the user activates the switch again. In some embodiments, switch 121 is a trigger switch that when activated continues stimulation until the trigger switch is activated again by the user.

Wearable 122 comprises an assortment of sensing and stimulating components, and can be in the form of an article of clothing or harness that can be worn by a subject to position the components adjacent to regions of sensing and stimulating interest. Wearable 122 comprises at least one electrode 124. The at least one electrode 124 includes stimulating electrodes and can also include sensing electrodes. Stimulating electrodes are configured to administer electrical stimulation, while sensing electrodes are configured to measure a physiological response. For example, sensing electrodes can include electrocardiography electrodes, electromyography electrodes, electroencephalography electrodes, and the like. In some embodiments, the stimulating electrodes are electrically linked to the sensing electrodes. In various embodiments, wearable 122 can further include one or more additional sensors, such as temperature sensors, blood pressure sensors, pulse oximetry sensors, glucose sensors, and the like. In some embodiments, wearable 122 comprises one or more switches for manual activation and deactivation of the abovementioned components. Wearable 122 can further be powered by a power source 126 (such as a battery or an electrical plug). In some embodiments wearable 122 can further include a wired or wireless transmitter 128 for transmitting data collected by the various electrodes and sensors, a wired or wireless receiver 130 for receiving instructions for activating stimulating electrodes, and a non-transitory computer-readable medium 132 connected to a processor to store data collected by the various electrodes and sensors.

Referring now to FIG. 1W, an exemplary wearable earpiece 150 is depicted. Earpiece 150 can have any suitable shape and fitting. For example, while earpiece 150 is depicted as having an in-ear canal piece, a concha cavum piece, and a concha cymba fin, it should be understood that earpiece 150 can comprise one or more of an in-ear canal piece, a concha cavum piece, a concha cymba fin, a behind-the-ear loop, an on-ear clip, and the like. Earpiece 150 comprises an outer surface having at least one conductive region. In some embodiments, the entirety of the outer surface is conductive. In some embodiments, specific portions of the outer surface are intentionally nonconductive whereas other specific portions of the outer surface are intentionally conductive. Earpiece 150 comprises at least one anode 152 and at least one cathode 154. In some embodiments, anode 152 and cathode 154 are provided as separate wearable components (as shown in FIG. 1F). For example, FIG. 1F depicts earpiece 150 as having conductive regions functioning as anode 152 and one or more additional electrodes or conductive elements as cathode 154. However, it should be understood that earpiece 150 can have conductive regions serving as cathode 154 and one or more additional conductive elements serving as anode 152, as well as any and all combinations thereof. In other embodiments, anode 152 and cathode 154 are both incorporated onto earpiece 150, wherein at least one first conductive region functions as anode 152 and at least one second conductive region functions as cathode 154. The conductive regions can be positioned on earpiece 150 to directly contact specific locations of an ear. The additional electrodes or conductive elements can be positioned on an ear, such as the tragus, or on any other part of a subject. In various embodiments, earpiece 150 can be wired or wireless.

Computer platform 134 comprises a wired or wireless transmitter 138 for transmitting instructions to wearable 122, and can also comprise a wired or wireless receiver 140 to collected data from bottle 102, wearable 122, or both, a non-transitory computer-readable medium 142 connected to a processor to store instructions and collected data, and can be powered by a power source 136 (such as a battery or an electrical plug).

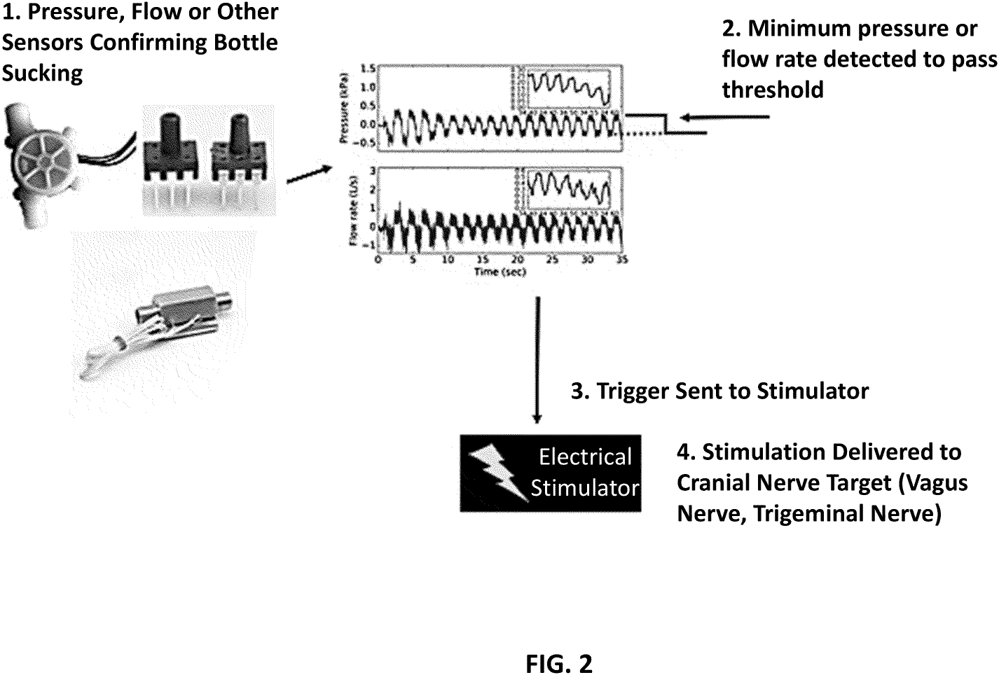

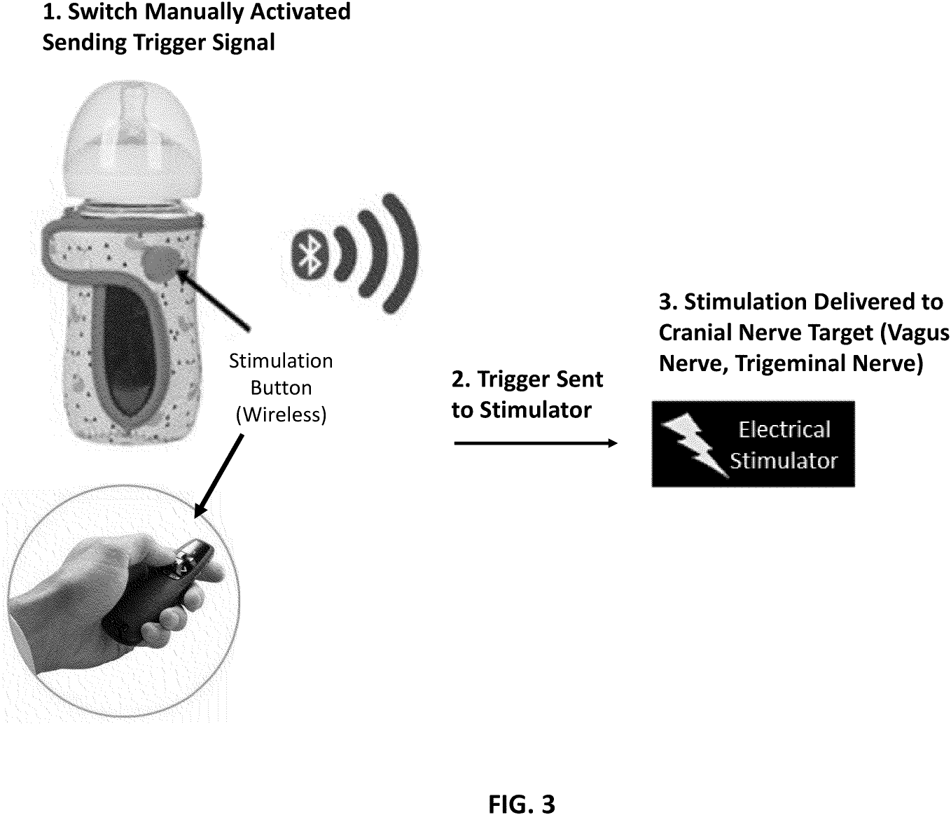

As described above, the various components of system 100 can be used alone or in combination to administer cranial nerve stimulation with or without feedback. In a non-limiting first example, bottle 102 is coupled with wearable 122. Bottle 102 can communicate with wearable 122 by way of transmitter 118 to receiver 130 that bottle 102 is in position for feeding. As shown in FIG. 2, bottle 102 can sense a minimum change in volume, flow, and/or pressure that passes a threshold to initiate a trigger. Bottle 102 communicates to wearable 122 to supplement feeding behavior by activating an electrode 124 adjacent to a cranial nerve, thereby stimulating the cranial nerve. Feeding behavior can be monitored and further verified by bottle 102. Feeding behavior can also be monitored and verified by an electrode 124 sensing cheek and jaw muscle activation. Feeding can continue by timing and synchronizing sensing of feeding initiation from bottle 102 and stimulation from wearable 122. As shown in FIG. 3, electrode 124 can also be activated manually, such as by using a manually activated switch 121. As described elsewhere herein switch 121 can be positioned in any location convenient for a user, such as in a handheld wireless device or integrated into a bottle 102 or sleeve covering.

In a non-limiting second example, wearable 122 can be used alone as a closed loop system. A sensing electrode 124 adjacent to one or more cheek and jaw muscles can be used to sense feeding initiation through a minimum change in muscle activation that passes a threshold to initiate a trigger. In response to the trigger, wearable 122 supplements feeding behavior by activating a stimulating electrode 124 adjacent to a cranial nerve. Feeding can continue by timing and synchronizing sensing of feeding initiation from a sensing electrode 124 and stimulation from a stimulating electrode 124. In this manner, wearable 122 functions as a closed loop system between sensing a minimum cheek and jaw muscle activation indicating feeding initiation and administering cranial nerve stimulation.

In other embodiments, activation of electrode 124 can be performed manually in an open loop configuration. For example, bottle 102, wearable 122, or both can each comprise one or more switches 121 as shown in FIG. 1C and FIG. 1D, wherein at least one switch is electrically connected to electrode 124 for manual activation, or is connected to a stimulating unit 133 (either wirelessly as shown in FIG. 1C or through a wired connection as shown in FIG. 1D) that is then connected to electrode 124. A user thereby initiates and regulates the rate of electrode 124 activation and subsequent feeding by the manual pressing of the at least one switch.

Stimulating unit 133 is configured to provide initialization, programming, monitoring, status reporting, etc. to the user. In one embodiment, stimulating unit 133 may comprise a user interface having keypads, a display and a processing unit (e.g., FIG. 1E). Stimulating unit 133 is configured to receive user inputs, such as through manipulation of keys of the keypad. In one embodiment, stimulating unit 133 may receive input by any other means known to one skilled in the art, such as by audio inputs. In one embodiment, stimulating unit 133 may provide output to a user by textual and graphical presentation on the display and/or by aural output through audio. In one embodiment, current manipulation may be done manually using the keys of the keypad. In one embodiment, the keypad comprises numbered keys from 0-9. Current can be changed manually by 0.1 mA increments from numbered buttons on the face of the device during stimulation in order to determine the appropriate treatment current via dose titration and also be able to be adjusted during the therapy session. One exemplary current stimulation setting is shown below in Table 1.

TABLE 1

Desired functionality of numbered buttons (i.e. current output)

| | |
|---|---|
| Numbered Buttons Pressed | Current Stimulation |
| “1” | 0.1 mA |
| “2” | 0.2 mA |
| “3” | 0.3 mA |
| ... | ... |
| “9” | 0.9 mA |
| “1” then “0” | 1.0 mA |
| “1” then “1” | 1.1 mA |
| ... | ... |
| “1” then “9” | 1.9 mA |
| “2” then “0” | 2.0 mA |

In one embodiment, the display may be suitable for displaying text characters. In one embodiment, the display may be adapted to provide gray scale display, such as 4 or 8 bit gray scale, and/or may provide color display, such as 16 bit color. In one embodiment, the display may include a back light for easy viewing in many light conditions.

In one embodiment, the display unit can let the user know that stimulation is on by any means known to one skilled in the art.

In one embodiment, stimulating unit 133 may further comprise a power switch and an indicator that indicates when the power is on. In one embodiment, stimulating unit 133 may further comprise an internal power supply, and an indicator for indicating the status of the internal power supply. Once the power switch is on, pressing keys on the keypad may enable current to be increased or decreased. This enables a patient threshold to be established.

In one embodiment, system 100 further comprises an impedance sensor. In one embodiment, impedance may be monitored and displayed on the display. In one embodiment, the processing unit can trigger an alarm for pre-set impedance limits. In one embodiment, the processing unit may be configured to shut off the system if impedance is not corrected by the user. In one embodiment, the processing unit may be configured to wait for about 30 seconds for impedance to be corrected before proceeding with the shut off. In one embodiment, the waiting period may be any time adjusted by the user. In one embodiment, the impedance sensor may be configured to let the user know whether simulating unit 133 has good, fair or poor contact with the patient.

The processing unit may be used to adjust the pulse characteristics. In one embodiment, the processing unit is able to allow selection of the pulse shape. In one embodiment, the pulse shape may be selected from the group including but not limited to a square wave, a mono-pulse and etc. In one embodiment, the processing unit may be able to allow selection of the pulse width. In one embodiment, pulse width may be ranging between approximately 20 - 500 microseconds. In one embodiment, pulse widths may be in the range of 10 - 1000 microseconds. The ranges of pulse widths made available according to embodiments of the invention may be adjusted based upon the particular type of stimulation being implemented. In one embodiment, pulse width may be 500 microseconds. In one embodiment, the pulse width may be selected from the group consisting of: nominal, 500 microseconds, settable from a range between 250 -500 microseconds. In one embodiment, the processing unit may be used to set the voltage. In one embodiment, the voltage may be a maximum of 40 V, delivering constant current. In one embodiment, the processing unit may be used to set the current. In one embodiment, the current may be between approximately 0-4 mA. In one embodiment, the current may be adjusted from 0-2 mA by increments of 0.1 mA. In one embodiment, the current may be adjusted by increments of more than 0.1 mA. In one embodiment, the current may be factory software adjustable up to 4 mA. In one embodiment, the system is configured to prevent stimulation above 2 mA. In one embodiment, the system possesses memory to store each individual patients calibrated stimulation level. In one embodiment, the system possesses memory to store each individual patients calibrated stimulation level and then disallows stimulation above that level. In one embodiment, the processing unit may be used to set the repetition rate. In one embodiment, the repetition rate may be 25 Hz. In one embodiment, the repetition rate may be less than 25 Hz. In one embodiment, the repetition rate may be more than 25 Hz.

In one embodiment, stimulating unit 133 may further comprise an additional port positioned anywhere on the unit. In one embodiment, the additional port may be connected to an external trigger switch. In one embodiment, when the external trigger switch is pressed, the trigger switch may run the pulse train for a defined period of time unless it is pressed again during that time interval and then it would turn the pulse train off. In one embodiment, the external trigger switch may manually enable activation for a defined period of stimulation of the pulse train and if pressed again, prior to the defined period of time automatic termination, may turn off the pulse train. In one embodiment, this defined period of time may be 30 seconds to 30 minutes. In one embodiment, this defined period of time is 30 second, 60 seconds, 120 seconds, 5 minutes, 10 minutes, 15 minutes, 20 minutes or 30 minutes. This gives the operator control of the triggering function and can also enable them to turn off the pulse train quickly, if need be. Alternatively, two switches capable of being mounted on the bottle could be used for the trigger, with one activating the pulse train, which would then run if not manually interrupted by the other switch.

In one embodiment, when the pulse train is triggered on, it will immediately deliver the current level on the display. In one embodiment, stimulating unit 133 may comprise an indicator light (or other notification) to alert the user that the device is providing stimulation current.

In various embodiments, system 100 can operate as a hybrid closed loop and open loop system. For example, system 100 can be initiated as described above and begin operation as a closed loop system, whereupon a user can override the closed loop operation with manual activation of electrode 124 to transition operation to an open loop system. At any point during open loop operation, the user can cease manual activation, whereupon the system can automatically return to closed loop operation. Likewise, system 100 can be initiated through direct user activation and begin operation as an open loop system. Cycling through at least one manual nerve stimulation and subsequent feeding initiation may permit the user to cease manual activation and allow closed loop operation to step in. In some embodiments, system 100 can have both open and closed loop capabilities. In other embodiments, system 100 has only one of the open or closed loop capabilities.

Computer platform 134 can be used to supplement communication between bottle 102 and wearable 122. Computer platform 134 can also be used to facilitate operation, monitoring, and data collection/storage for bottle 102, wearable 122, or both. In some embodiments, computer platform 134 can be used to adjust the timing and intensity of electrode stimulation in wearable 122 according to data received from bottle 102, wearable 122, or both. In some embodiments, the timing and intensity of electrode stimulation in wearable 122 is adjusted automatically to maintain measurable parameters within thresholds set by computer platform 134. Measurable parameters include but are not limited to heart rate, blood pressure, muscle activation rate, neural patterns, bottle volume, bottle position, and the like. In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

It should be understood that the components of system 100 are not limited to use in training feeding behavior and can be used to enhance infant development in a variety of manners. In some embodiments, cranial nerve stimulation is effective in neurodevelopment and neuro-rehabilitation by increasing brain white matter integrity and inter-regional communication among the various regions of the brain. For example, treatment can have neuroplastic effects such as increasing white matter plasticity as measured by fractional anisotropy. In some embodiments, microstructural changes in white matter, such as increased organization, packing, and membrane integrity, reflect improved oromotor function in infants. In some embodiments, white matter tracts that experience increased tissue anisotropy after treatment include but are not limited to: the anterior limb of internal capsule, inferior front-occipital fasciculus, external capsule, and superior longitudinal fasciculus. In some embodiments, cranial nerve stimulation is effective in enhancing motor function, such that activities including head lifting, rolling, sitting up, gripping, lifting, throwing, crawling, walking, climbing, and descending can be trained and improved. In some embodiments, cranial nerve stimulation is effective in modulating behavior. Behavior modulation can include positive reinforcement for good behavior, negative reinforcement for bad behavior, and the reduction or treatment of neurological and psychological disorders or injury.

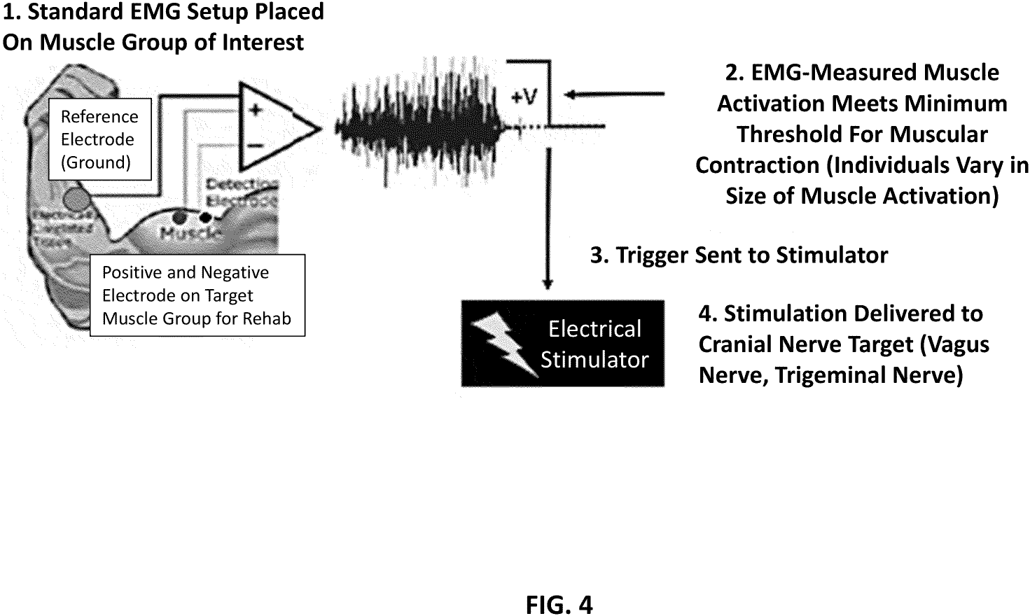

It should be understood that the components of system 100 are not limited to use in infants and can be used in children, adults, and the elderly. In various embodiments, the components of system 100 are further applicable to animals, including mammals, reptiles, birds, fish, and the like. In some embodiments, cranial nerve stimulation is effective in treating muscle-related disorders and rehabilitation, such as post-stroke upper and lower motor limb rehab paradigms, wherein muscle groups involved in specific rehabilitation paradigms are targeted. For example, referring now to FIG. 4, components of system 100 (such as a sensing electrode 124 on wearable 122) can measure muscle activation in one or more muscle groups of interest that passes a minimum threshold to initiate a trigger. Wearable 122 can supplement muscle activation by activating a stimulating electrode 124 adjacent to a cranial nerve, thereby stimulating the cranial nerve. Further activation of the one or more muscle groups of interest can be monitored and verified by a sensing electrode 124. Muscle activation can continue by timing and synchronizing sensing of muscle activation initiation from a sensing electrode 124 and stimulation from a stimulating electrode 124, such as in a closed loop system. In some embodiments, cranial nerve stimulation is effective in modulating muscular or neural diseases or disorders, including but not limited to Parkinson’s disease, dyskinesia, dystonia, and the like.

### Cranial Nerve Stimulation Methods

The present invention is also based in part on methods for administering noninvasive cranial nerve stimulation. As described elsewhere herein, the methods are effective in enhancing rehabilitation and recovery by improving neuroplasticity and coupling muscle training with feedback.

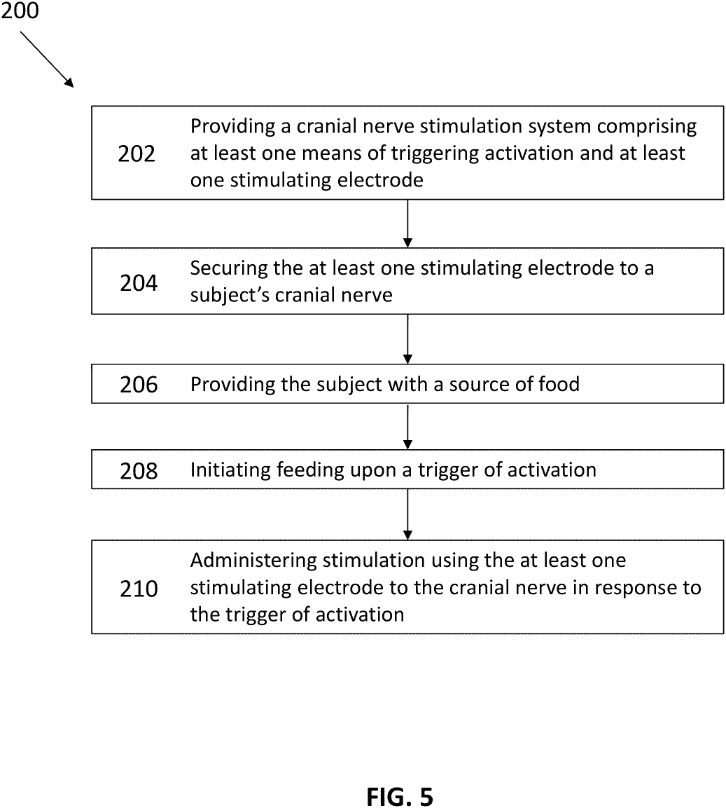

In some embodiments, the methods relate to enhancing oromotor skills. Referring now to FIG. 5, an exemplary method 200 is depicted. Method 200 begins with step 202, wherein a cranial nerve stimulation system is provided, the system comprising at least one means of triggering activation and at least one stimulating electrode. The at least one means of triggering activation can be, but is not limited to one of the following: a sensing electrode noninvasively secured adjacent to a subject’s cheek or jaw muscle, a sensing mechanism integrated into a nipple or bottle used in feeding, or a manual switch. In step 204, at least one stimulating electrode is noninvasively secured adjacent to a subject’s cranial nerve. In step 206, the subject is provided with a source of food. In step 208, feeding is initiated upon a trigger of activation. A trigger of activation may depend on the at least one means of triggering activation provided, and can occur when: a sensing electrode senses muscle activation that surpasses a minimum threshold, a sensing electrode senses some displacement of food, or a manual switch is activated. In step 210, stimulation is administered using the at least one stimulating electrode to the cranial nerve in response to the trigger of activation.

In some embodiments, the subject is an infant, and the oromotor skills relate to suckling. In various embodiments, the cranial nerve can be selected from the group consisting of the trigeminal nerve, the facial nerve, the accessory nerve, the hypoglossal nerve, the auricular branch of the vagus nerve, the main bundle of the vagus nerve, and the like. In various embodiments, the electrodes are noninvasively secured using an adhesive, a clip, a patch, an ear plug, a head band, a neck brace, a collar, a head covering, and the like. In some embodiments, the steps are performed in the recited order. In various embodiments, step 208 and step 210 are repeated in a closed loop system. In some embodiments, step 210 is initiated by a user in an open loop system. In an open loop system, stimulation of the cranial nerve is performed manually.

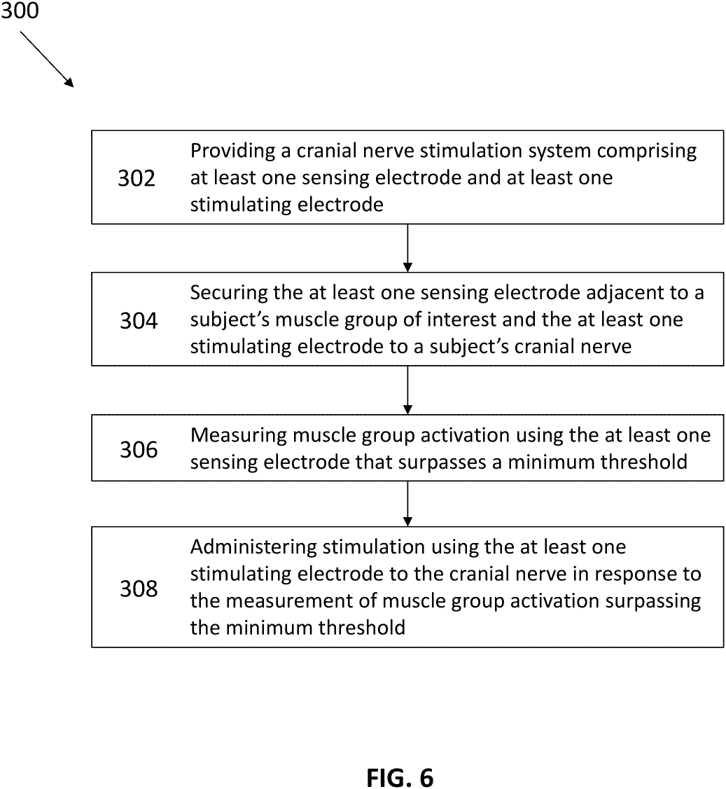

In some embodiments, the methods relate to muscle rehabilitation. Referring now to FIG. 6, an exemplary method 300 is depicted. Method 300 begins with step 302, wherein a cranial nerve stimulation system is provided, the system comprising at least one sensing electrode and at least one stimulating electrode. In step 304, the at least one sensing electrode is noninvasively secured adjacent to a subject’s muscle group of interest, and the at least one stimulating electrode is noninvasively secured adjacent to a subject’s cranial nerve. In step 306, muscle group activation is measured using the at least one sensing electrode that surpasses a minimum threshold. In step 308, stimulation is administered using the at least one stimulating electrode to the cranial nerve in response to the measurement of muscle group activation surpassing the minimum threshold.

In various embodiments, the cranial nerve can be selected from the group consisting of the trigeminal nerve, the facial nerve, the accessory nerve, the hypoglossal nerve, the auricular branch of the vagus nerve, the main bundle of the vagus nerve, and the like. In various embodiments, the electrodes are noninvasively secured using an adhesive, a clip, a patch, an ear plug, a head band, an arm band, a brace, a collar, a wrapping, and the like. In some embodiments, the steps are performed in the recited order. In various embodiments, step 306 and step 308 are repeated in a closed loop system. In some embodiments, step 308 is initiated by a user in an open loop system. In an open loop system, stimulation of the cranial nerve is performed manually, which may render step 306 to be optional.

In various embodiments, the methods of the present invention select certain minimum thresholds of muscle activation. In some embodiments, the methods select for a minimum threshold of muscle activation that is determined by an absolute measurement. For example, the minimum threshold of muscle activation can be selected from an absolute value of about 0.1 µV, 0.5 µV, 1 µV, 5 µV, 10 µV, 50 µV, 100 µV, 200 µV, 300 µV, 400 µV, 500 µV, 1 mV, 5 mV, 10 mV, 20 mV, 30 mV, 40 mV, or 50 mV. In some embodiments, the methods select for a minimum threshold of muscle activation that is determined by a change from a base measurement taken at rest. For example, the minimum threshold of muscle activation can be selected from an increase or decrease of about 0.1 µV, 0.5 µV, 1 µV, 5 µV, 10 µV, 50 µV, 100 µV, 200 µV, 300 µV, 400 µV, 500 µV, 1 mV, 5 mV, 10 mV, 20 mV, 30 mV, 40 mV, or 50 mV. In some embodiments, the methods select for a minimum threshold of muscle activation that is determined by a percentage of a typical maximum potential of the muscle. For example, the minimum threshold of muscle activation can be selected from about 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of the typical maximum potential of the muscle.

In various embodiments, the methods of the present invention select certain parameters for cranial nerve stimulation. In some embodiments, the methods select for an intensity of stimulation. For example, the intensity of stimulation can be selected from about 0.01 mA, 0.05 mA, 0.1 mA, 0.2 mA, 0.3 mA, 0.4 mA, 0.5 mA, 0.6 mA, 0.7 mA, 0.8 mA, 0.9 mA, 1 mA, 1.5 mA, 2 mA, 2.5 mA, 3 mA, 3.5 mA, 4 mA, 4.5 mA, 5 mA, 6 mA, 7 mA, 8 mA, 9 mA, or 10 mA. In some embodiments, the method can have a hard limit for maximum stimulation intensity which cannot be exceeded. In some embodiments this hard limit for maximum stimulation intensity is 0.1 mA, 0.2 mA, 0.3 mA, 0.4 mA, 0.5 mA, 0.6 mA, 0.7 mA, 0.8 mA, 0.9 mA, 1 mA, 1.5 mA, 2 mA, 2.5 mA, 3 mA, 3.5 mA, 4 mA, 4.5 mA, 5 mA, 6 mA, 7 mA, 8 mA, 9 mA, or 10 mA. In some embodiments the methods select for a frequency of stimulation. For example, the frequency of stimulation can be selected from about 1 Hz, 2 Hz, 3 Hz, 4 Hz, 5 Hz, 6 Hz, 7 Hz, 8 Hz, 9 Hz, 10 Hz, 15 Hz, 20 Hz, 25 Hz, 30 Hz, 35 Hz, 40 Hz, 45 Hz, or 50 Hz. In some embodiments, the methods select for a pulse width of stimulation. For example, the pulse width of stimulation can be selected from about 10 µs, 20 µs, 30 µs, 40 µs, 50 µs, 60 µs, 70 µs, 80 µs, 90 µs, 100 µs, 150 µs, 200 µs, 250 µs, 300 µs, 350 µs, 400 µs, 450 µs, 500 µs, 550 µs, 600 µs, 650 µs, 700 µs, 750 µs, 800 µs, 850 µs, 900 µs, 950 µs, or 1 ms. In some embodiments, the methods select for a duration of stimulation on and off periods. For example, the duration of stimulation on and off periods can be selected from about 0.1 seconds, 0.5 seconds, 1.5 seconds, 2 seconds, 2.5 seconds, 3 seconds, 3.5 seconds, 4 seconds, 4.5 seconds, 5 seconds, 10 seconds, 20 seconds, 30 seconds, 40 seconds, 50 seconds, 1 minute, 2 minutes, 3 minutes, 4 minutes, 5 minutes, 10 minutes, 15 minutes, 20 minutes, 30 minutes, 40 minutes, 45 minutes, 50 minutes, and 1 hour. The on and off periods can have the same duration or different durations.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

### Example 1: How to Measure a Baby’s Suck? Closing the Loop on Transcutaneous Auricular Vagus Nerve Stimulation (taVNS) to Enhance Oromotor Development of Impaired Infants: Which Electrode is Best?

Feeding difficulty due to oromotor dyscoordination is a primary concern for infants who are born preterm or suffer hypoxic ischemic encephalopathy (HIE). Vagal Nerve Stimulation (VNS) can increase neural plasticity, and when paired with rehabilitation, can enhance motor learning. Recently, it was demonstrated that non-invasive VNS can be accomplished via electrical stimulation of the auricular branch of the vagus nerve using a new method called transcutaneous auricular vagus nerve stimulation (taVNS). The goal of the present study is to develop a closed-loop automatic system that pairs taVNS with muscle activation from sucking, using electromyography (EMG) as a trigger. This system may allow better suck and stimulus pairing that is also less labor-intensive.

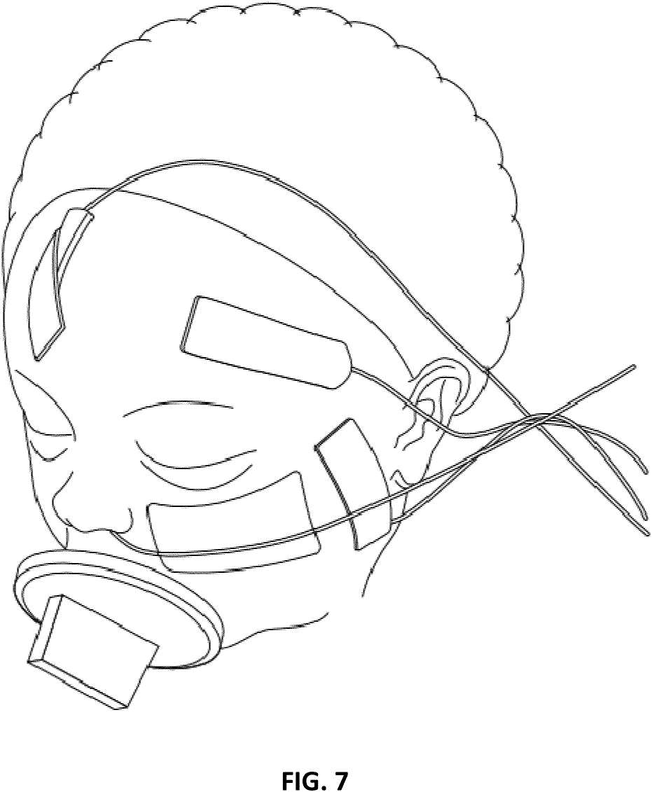

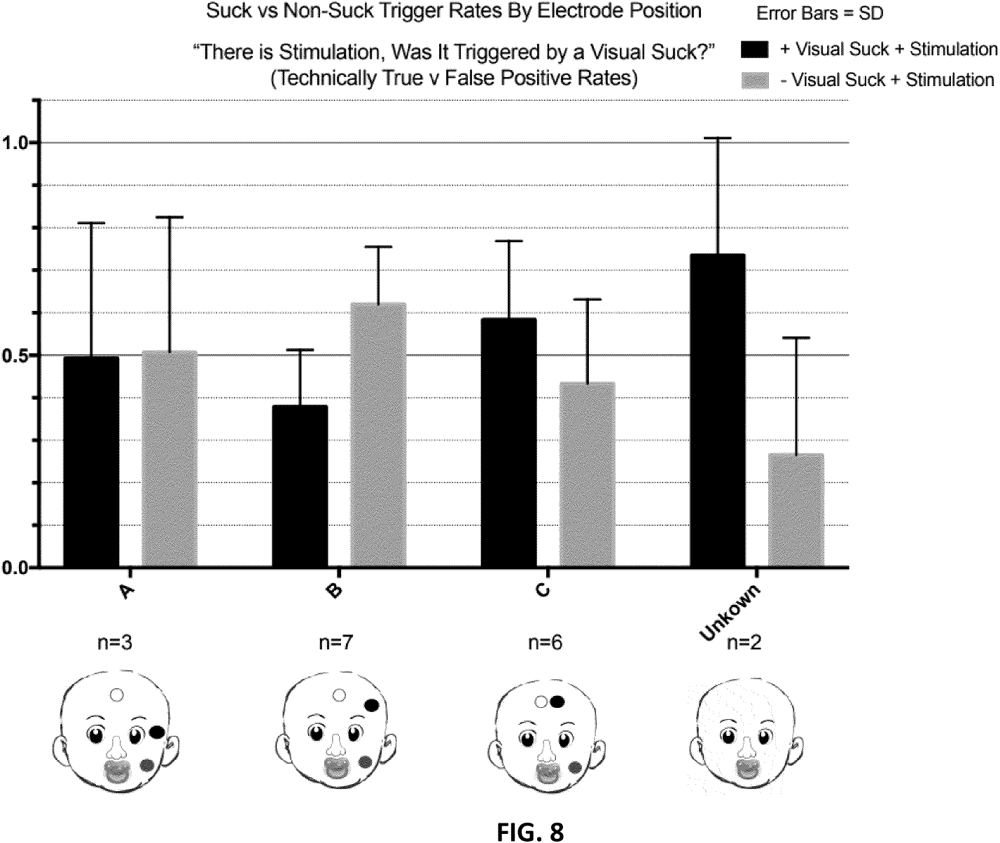

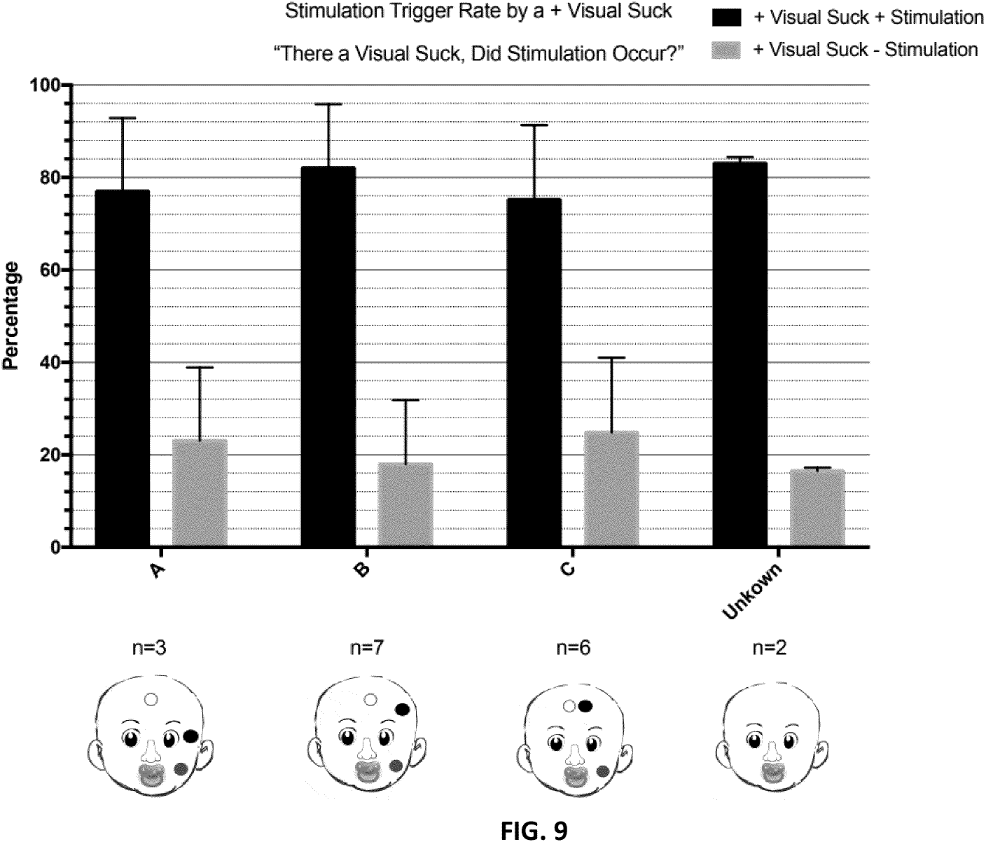

These investigations were designed to test the best location for reference electrode placement and the fidelity of stimulation paired with sucking. Three different EMG electrode placements (A, B, C) were compared to optimize the specificity and sensitivity of the automated system in 2 pre-term neonates enrolled in a larger pilot trial (example shown in FIG. 7). Triggered stimulation was delivered using a left ear electrode at 0.1 mA below perceptual threshold, 25 Hz frequency, 500 µs pulse width, for a 3.5 second train. The primary outcomes of this study were specificity (stimulations correctly paired to a visual suck, FIG. 8) and sensitivity (visual sucks that triggered or occurred during stimulation, FIG. 9).

Locations A, B, and C had a mean specificity of 49.3±31.8 (n=3), 37.9±13.4 (n=7), and 58.3±18.5 (n=6), respectfully. Locations A, B, and C had a mean sensitivity of 77±15.9 (n=3), 82±13.8 (n=7), and 75.2±16.2 (n=6), respectively. Electrode placement C was feasible and better tolerated. The placement produced the highest average (60%) rate of stimulation induced by a real visual suck while minimizing stimulation triggered by non-visual suck (40%). All placements seemed to perform equally at a rate of about 77-81% triggers induced by a visual suck.

These results demonstrate that EMG electrode position C was the most efficient with 58% of stimulation trains correctly pairing with visual sucks while maintaining good sensitivity to visual sucks. Using EMG in a closed-loop taVNS system is a safe and effective way to trigger taVNS stimuli in infants.

### Example 2: Treating Neonates With Cranial Nerve Stimulation

In preterm infants with brain dysmaturation or term infants with hypoxic ischemic encephalopathy (HIE), feeding difficulty is the primary reason for delayed hospital discharge. Failure to achieve full oral feedings may be due to closure of critical developmental windows of neuroplasticity, or due to overt brain injury in HIE infants. Current therapies are limited to feeding by occupational or speech therapists once a day, and gastrostomy tube (g-tube) placement.

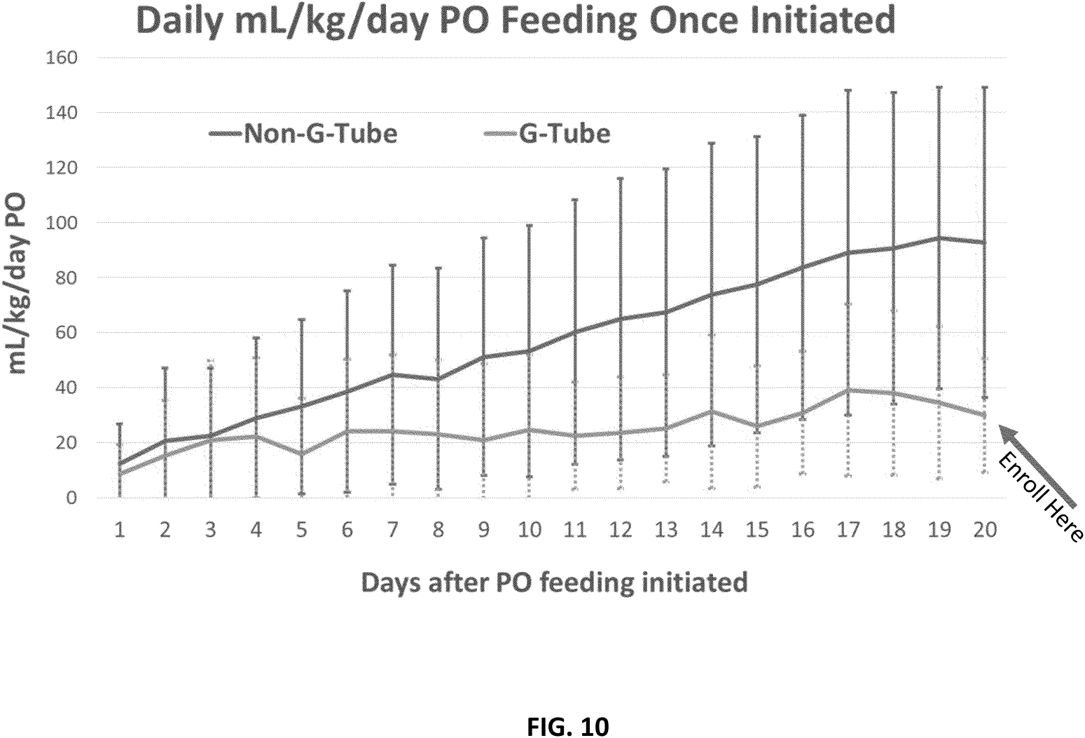

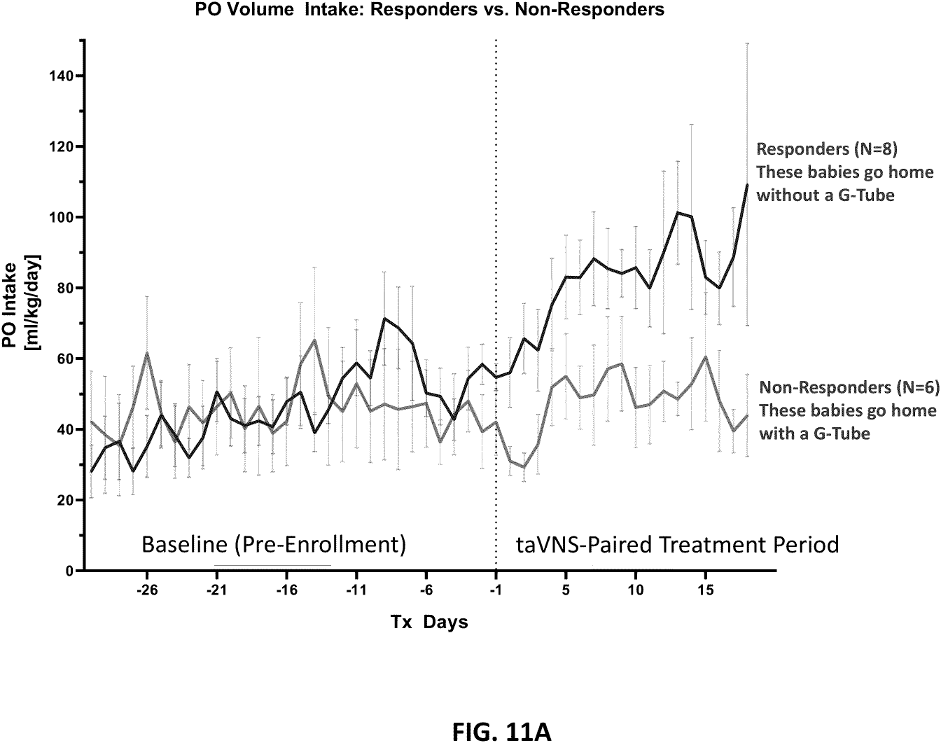

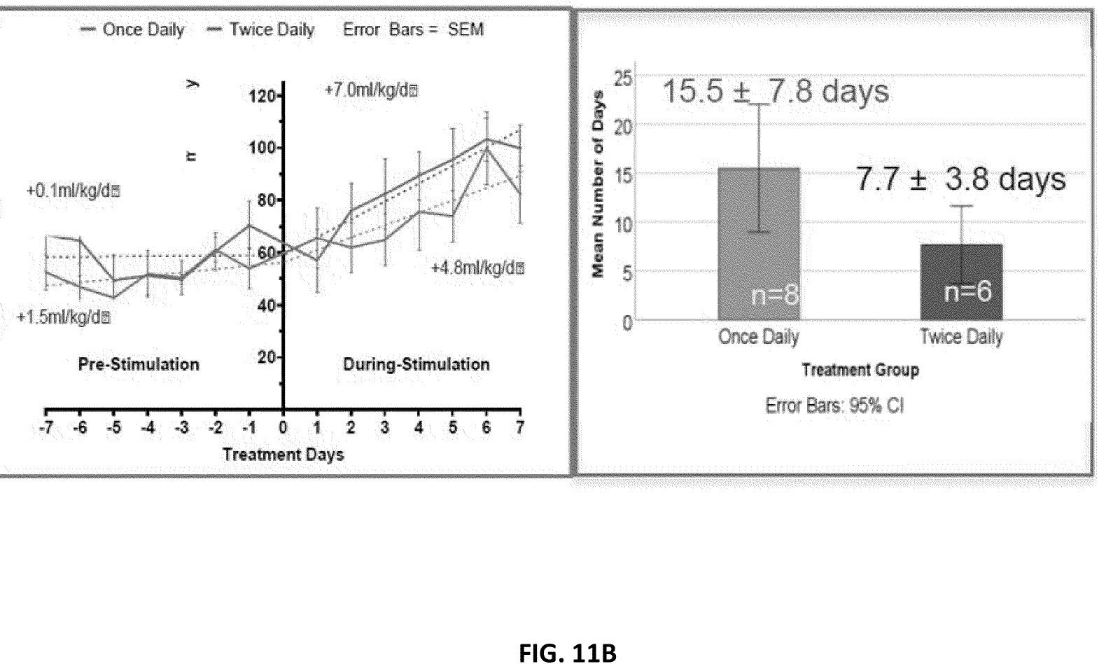

The present study monitored intake of infants 20 days post-oral (PO) feeding initiation. Infants that have failed feeding on average for 49 days were determined to be g-tube candidates and were enrolled in the cranial nerve stimulation trial (FIG. 10). 14 babies were analyzed in an interim analysis (FIG. 11A). All babies were g-tube candidates and had been attempting to feed orally for an average of 49 days before enrollment. Treatment was administered based on previous protocols (stimulation delivered using a left ear electrode at 0.1 mA below perceptual threshold, 25 Hz frequency, 500 µs pulse width, for a 3.5 second train). 57% of the babies (8 of 14) reached the adequate PO intake (full feeds orally) that is clinically required to be discharged without a g-tube. The results demonstrate that in more than half of babies, cranial nerve stimulation facilitates their rehabilitation, enhances neuroplasticity, and facilitates motor learning. FIG. 11B further demonstrates a potential dose response with a significantly greater increase in slopes of daily PO feeding volumes (F, 8.05, p = 0.01, FIG. 11B left) and shorter time to full PO feeds (p < 0.05, FIG. 11B right) in twice versus once daily treatments.

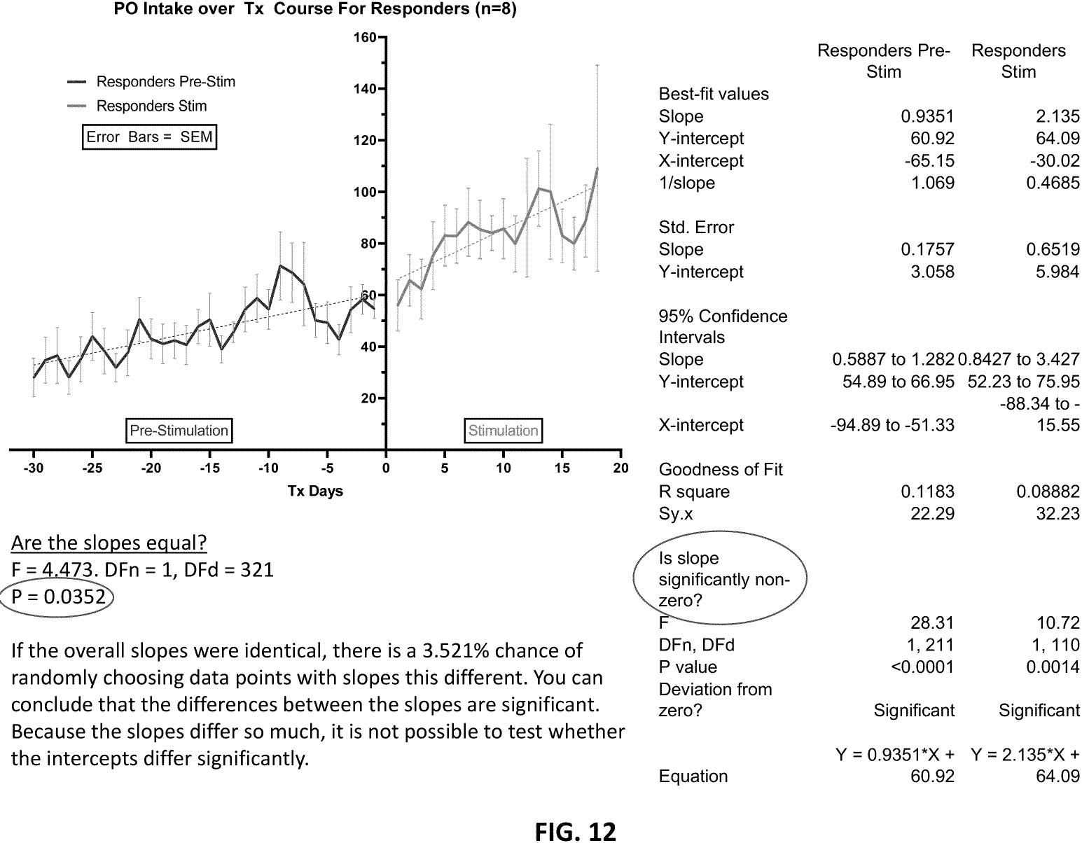

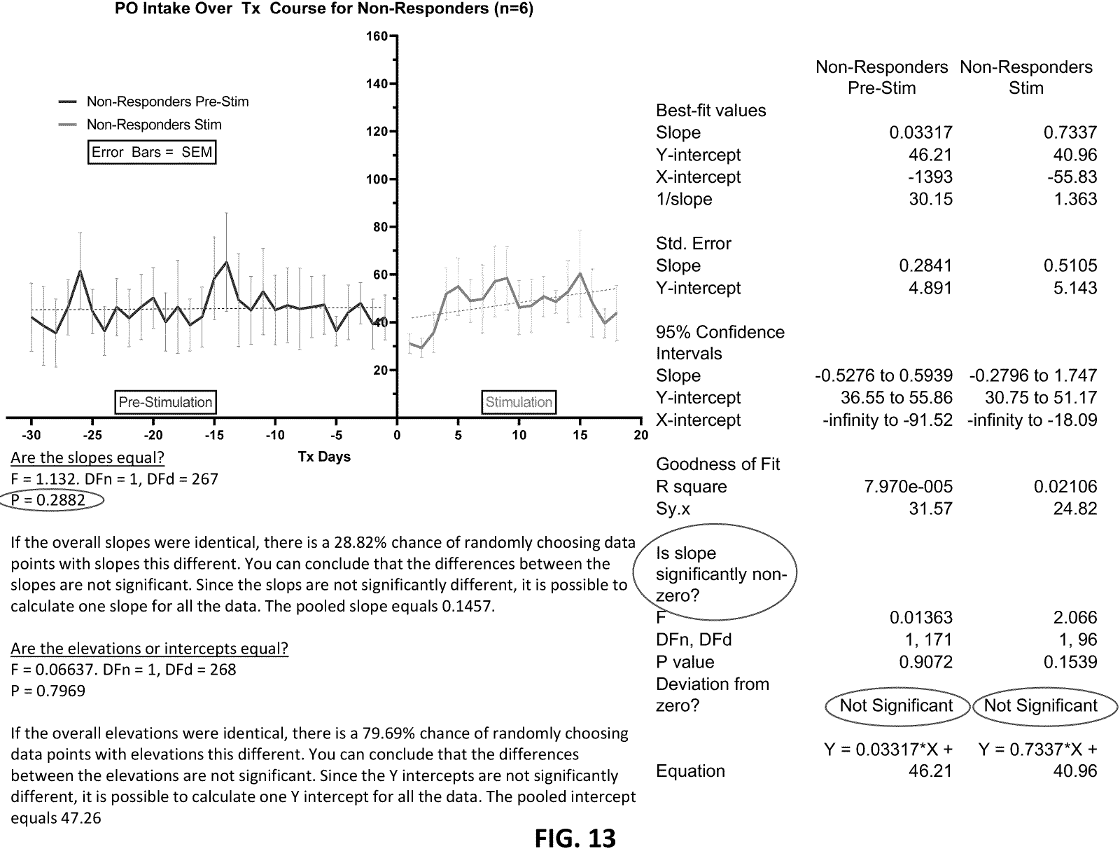

FIG. 12 and FIG. 13 depict the statistical analysis of the responder group and non-responder group. FIG. 12 shows that linear regression comparison of responders before and during stimulation treatment are significantly different, such that the slope increases after treatment. FIG. 13 shows that linear regression comparison of non-responders before and during stimulation treatment are not significantly different.

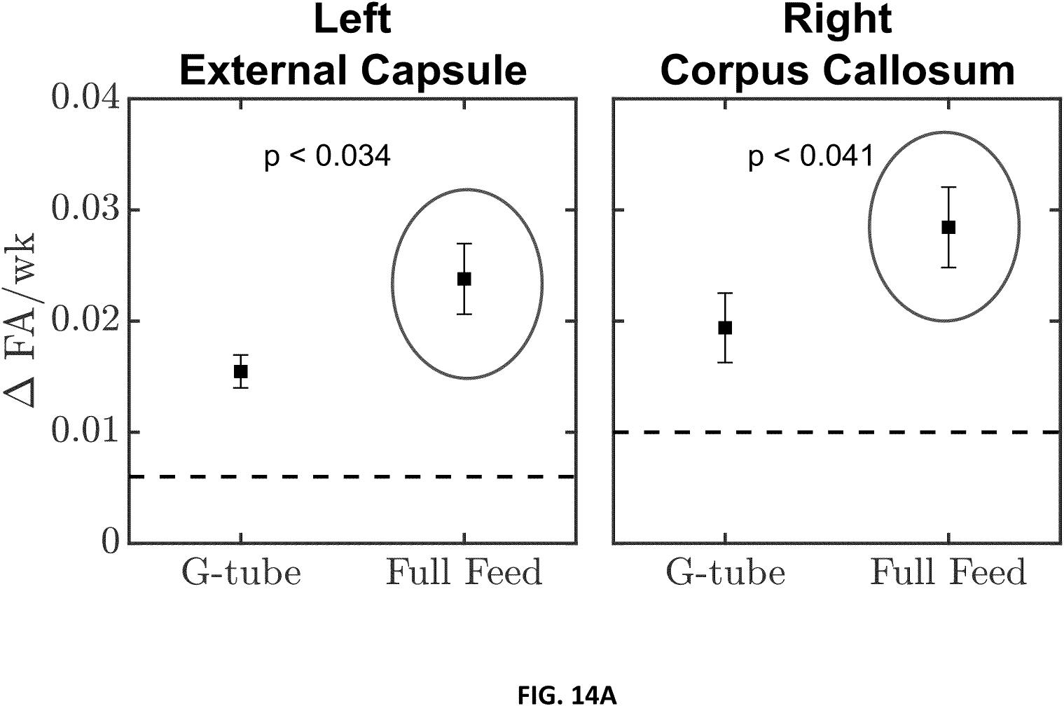

Treatment candidates were imaged to monitor the effects of treatment on brain development. Babies were scanned using MRI, treated for 2-4 weeks, and scanned again to investigate changes in white matter tracts. FIG. 14A and FIG. 14B demonstrate that cranial nerve stimulation had a greater effect on brain white matter tract integrity as indicated by fractional anisotropy (FA) and axial kurtosis (K_{||}) in the responder group (full feed) than in the non-responder group (g-tube). Specific white matter tracts related to motor and sensorimotor integration were all strengthened. Furthermore, FA changes in both responder and non-responder groups were greater than expected with normal development (FIG. 14A), demonstrating that there is more inter-regional communication across the brain tract.

### Example 3: taVNS Paired With Oromotor Feeding Rehabilitation

The materials and methods are now described.

### Study Design

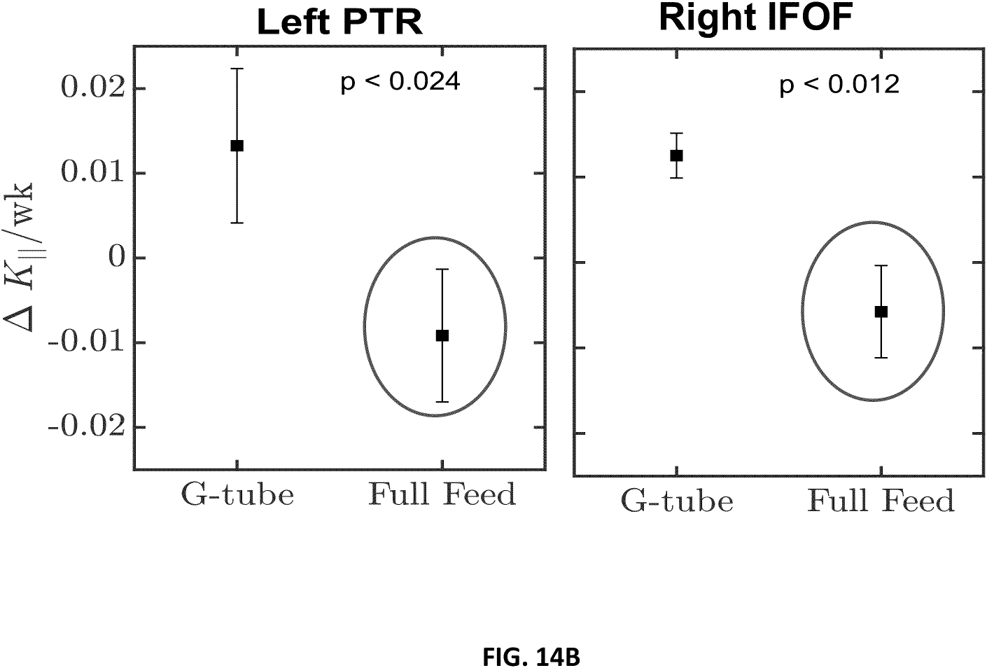

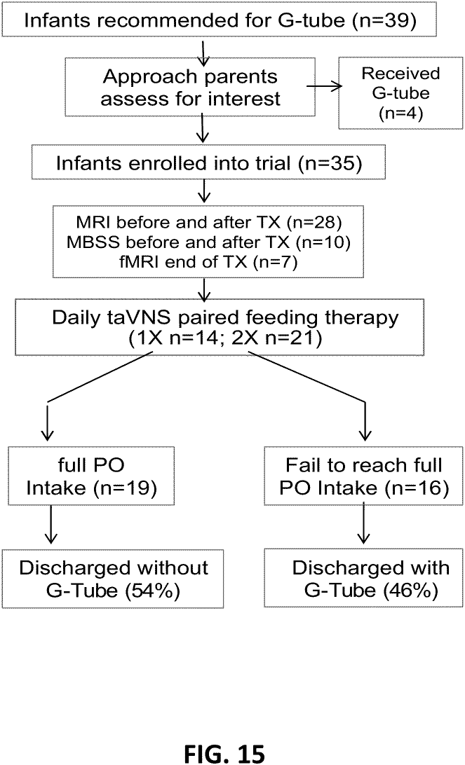

A pilot study was conducted, as a prospective, first-in-neonates/infants, phase 0, open-label trial with parental consent to assess the feasibility of combining taVNS with bottle feeding (FIG. 15). IRB approval was obtained with medical device designation of non-significant risk. The pilot trial had a 90% consent rate.

### Inclusion Criteria

Infants born ≤ 33 completed weeks gestation or any GA with HIE, brain injury; clinically stable, without significant respiratory support; failing to make progress with feeds in spite of usual interventions, with clinical planning and discussions with parents for G-tube.

### Exclusion Criteria

Cardiomyopathy, significant respiratory support, inability to attempt every feed by mouth.

### Primary Efficacy Outcomes

Targeted motor learning, measured by daily oral (per os, hereinafter “po”) feeding volumes before and during treatment; achievement of full oral feeds (>120 ml/kg/d with weight gain); avoidance of G-tube.

### Secondary Efficacy Outcomes

Brain changes as measured by improved integrity by diffusion imaging (DKI) in white matter tracts (WM) before and after a 2-week course of taVNS-paired feeding, normalized for weeks of development; Improvement in head and neck control and overall functional movements by STEP early developmental testing before and after taVNS-paired oromotor feeding treatment.

### Primary Safety Outcomes

Bradycardia (HR<80 bpm for 5 seconds), potential effect of taVNS on the dorsal nucleus and efferent cardiac vagus nerve fibers; skin irritation; discomfort measured by Neonatal and Infant Pain scale (NIPS) >3 point change, equivalent to pain with blood drawing.

### Secondary Safety Outcomes

Videofluoroscopic Barium Swallow (VFSS): VFSS was performed before beginning taVNS-paired bottle feeding and after the treatment course. During these studies, taVNS was turned on for 10 swallows during the VFSS and turned off for another 10 swallows, according to block randomization. The 8 infants scored to date typically had 3 studies of 20 swallows each condition (Thin-slow, Thin-fast, Thick).

### Treatment Protocol:

taVNS pulses delivered via a left ear electrode at frequency of 25 Hz, pulse width of 500 microseconds.
Perceptual threshold (PT) determined by increasing current by 0.1 mA until signs of discomfort.
taVNS delivered at 0.1mA less than the PT, paired with suck-swallowing during one (n=14) or two (n=13) 30-min oral feedings per day, for 2-3 wks.

The results are now described.

### Primary Efficacy Outcomes

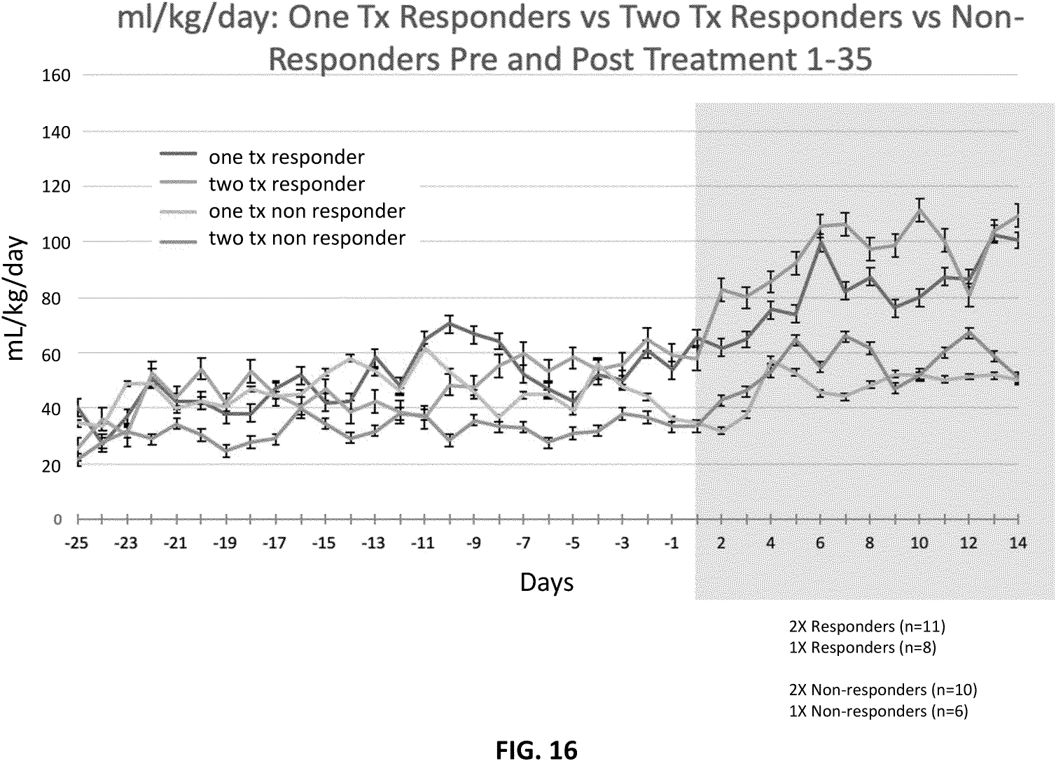

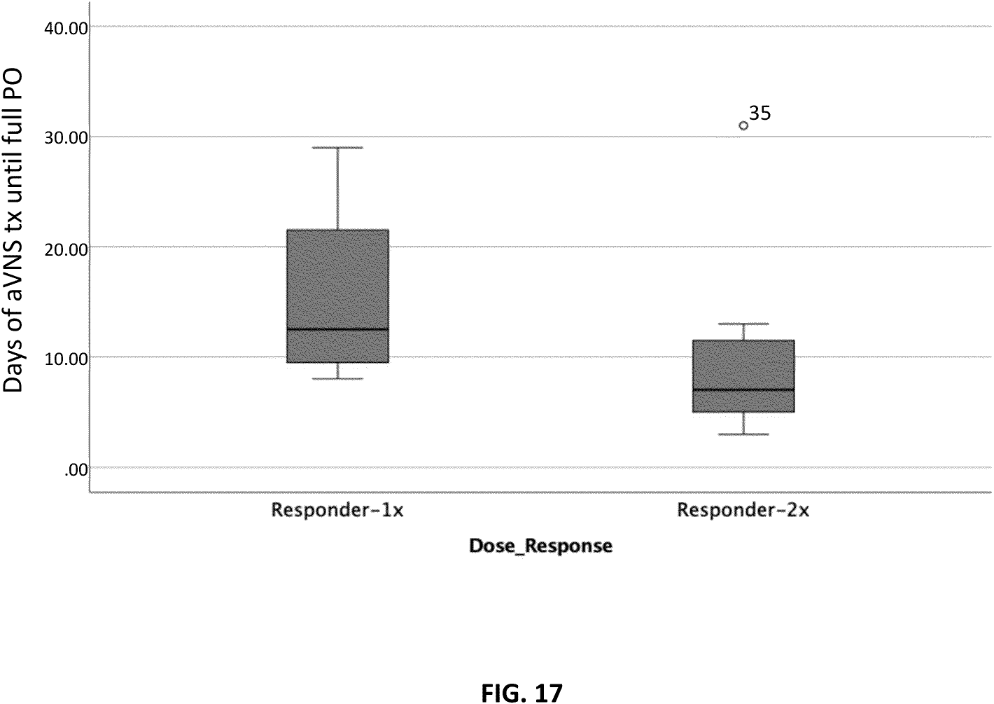

1. Achievement of full oral feeds (.120 ml/kg/d with weight gain); avoidance of G-tube.
54% or 19/35 infants destined for G-tube attained full oral feeds with taVNS paired feedings (Responders) and 16/35 were discharged home on G-tube feeds (Non-Responders).
Responders showed a significant increase in mean daily po volumes (ml/kg/d by mouth) with taVNS treatment vs Non-Responders who received G-tube (p<0.05, FIG. 16). Feeding volumes required for discharge are >120 ml/kg/d po with weight gain.
A dose response was demonstrated with significantly greater increase in slopes of daily po feeding volumes (F, 8.05, p=0.01, FIG. 16) and significantly shorter time to full po feeds (p<0.05, FIG. 17) in twice versus once daily treatments. Responders who received 1X daily taVNS-paired feeds achieved full oral feeds in a mean of 15.5 ± 7.8 days (n=8, 95% CI— 9.0, 22.0), while Responder infants who received 2X daily taVNS-paired feeds did so in 9.6 ± 7.8 days (n=11, 95% CI— 4.4, 14.9).

### Secondary Efficacy Outcomes

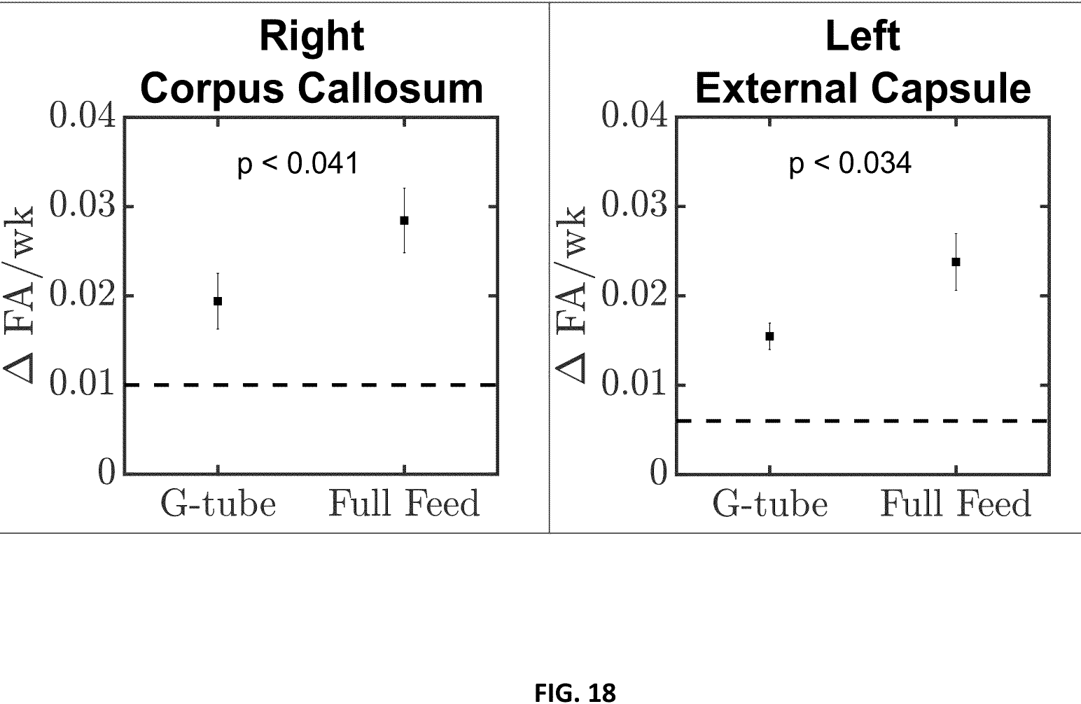

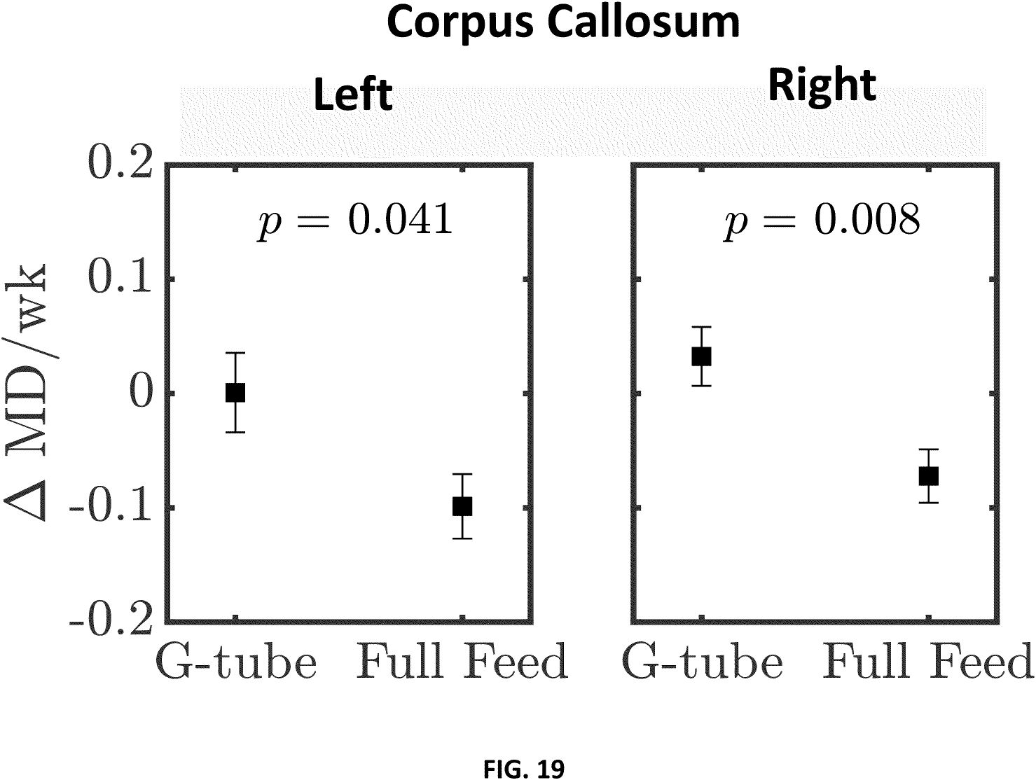

1. Diffusion MRI changes with taVNS-paired feeding: Diffusion imaging shows significantly different neuroplastic effects in taVNS responders vs non-responders.
Diffusion MRIs compared from pre- to post-taVNS treatment, show white matter plasticity changes in fractional anisotropy (FA) that are significantly greater for Responders versus Non-Responders in corpus callosum and external capsule, per week of development (FIG. 18, n=16). Both groups showed greater than normal FA increase, compared with published normal developmental changes in these tracts (indicated by dashed line).
In FIG. 19, the change in mean diffusivity from pre- to post-taVNS was significantly lower (n=18, p< 0.05) in the infants who achieved full po feeds (n=9) in the corpus callosum in both hemispheres than those who required a G-tube (n=9). FA indicates directional movement of water along axonal bundles, and indicates robustness of those white matter tracts, in increased organization, packing, and membrane integrity, even without myelination. Mean diffusivity indicates the extent of water movement out of the membrane in all directions and is generally higher with brain injury, neuroinflammation, and white matter dysmaturity.

Thus, taVNS paired with oromotor task of feeding results in white matter microstructural changes that correspond to improved oromotor function in infants. This provides proof of concept that taVNS paired with motor learning in young infants is safe and works by inducing neuroplastic changes in specific WM tracts involved in oral sensorimotor coordination.

2. Improvement in Head and Neck Control and Overall Functional Movements

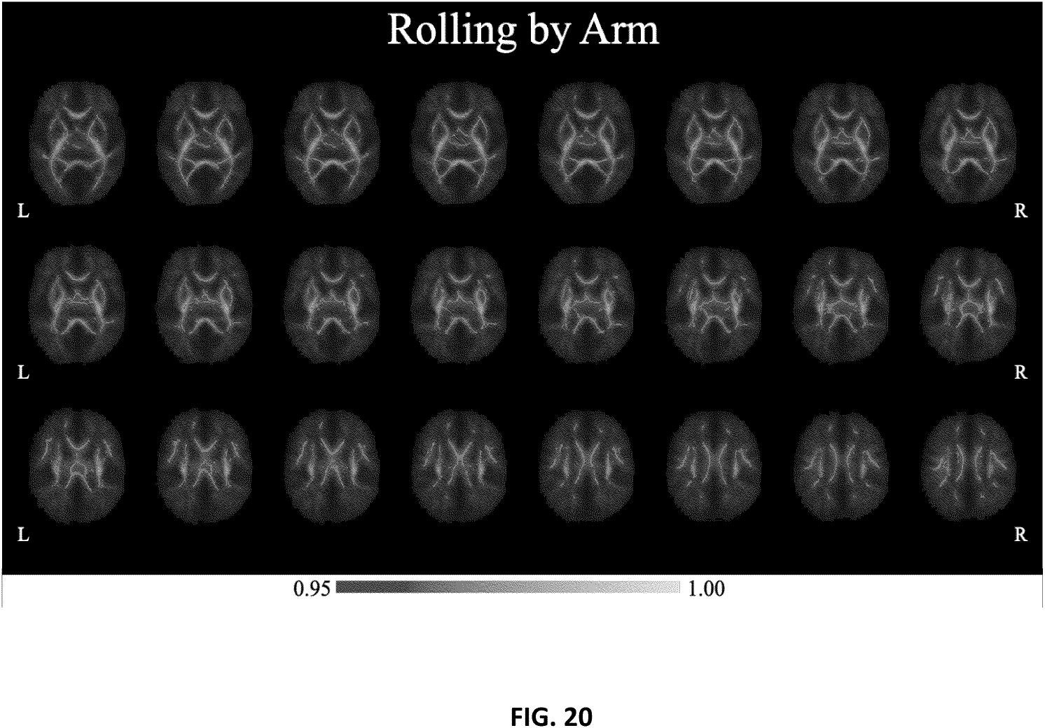

Early motor abilities were measured via the Specific Test of Early Infant Motor Performance (STEP), a ten-minute assessment of tone and movement performed at term age equivalent or 3 months corrected age, which was shown to predict 12-month motor and cognitive outcomes. The STEP was administered by the same trained pediatric occupational therapists before the start of taVNS intervention (pre-STEP) and upon completion of the intervention (post-STEP) for a total of 7 Responders and 12 Non-Responders with both pre- and post-taVNS-paired feeding STEP scores.
Infants who were responders had a significantly greater increase in total STEP score per week (mean 1.7 ± 2.1) from pre-to post-taVNS treatment than infants who were non-responders and required a G-tube (0.0 ± 0.85, p=0.04, paired t-test). The expected change in STEP score with development is 0.5 points/week. Therefore, taVNS-paired oromotor feeding training improves overall motor function in these at-risk infants.
When parsing out which types of movements were more different between pre- and post-taVNS paired feeding treatment, we found that Responders had significantly greater increase in four specific STEP items directly related to movements in the head and neck compared with non-responders. These included: head in supine with visual stimulation(p=0.01), head in supine with no visual stimulation (p=0.03), rolling elicited by arm(p=0.04), and head movements in supported sitting(p=0.01). Therefore, taVNS paired with bottle feeding for 2-3 weeks appears to primarily impact motor movements of the head and neck. Bottle-feeding involves coordination of 22 oral-facial muscles for successful intake, and the muscles of the head and neck are also activated during feeding. These muscles are critical for development as they permit the infant to explore their world both visually and from sensorimotor standpoint. These data demonstrate their function may be augmented by taVNS paired with the activity of bottle feeding.
In infants who performed well on one particular head and neck control item, Rolling by Arm, significantly greater change was found in fractional anisotropy within multiple WM regions (p< 0.05) after taVNS treatment in infants compared to infants who performed poorly (FIG. 20).

As STEP total and Rolling item scores were not significantly different pre-taVNS paired bottle feeding, it is likely that taVNS paired with bottle feeding facilitated integration of motor circuits. This study provides proof-of-concept that early neuromodulation paired with the motor task of feeding in at-risk infants may have positive effects on motor movements required for head and neck control.

### 3) Improvement in Video-Fluoroscopic Barium Swallow (VFSS)

VFSS was performed before beginning taVNS-paired bottle feeding and after the treatment course. During these studies, taVNS being turned on during the VFSS and turned off, according to block randomization, within feeding types of thin feeds with slow or faster nipple flow rates, and thickened feeds with larger nipple opening to allow flow. The 8 infants scored to date typically had 3 studies of 20 swallows (Thin-slow, Thin-fast, Thick). The Speech pathologists (SLP) performing and scoring VFSS with the Modified Barium Swallow Impairment Profile was blinded to taVNS on/off condition. Two SPL scored each study independently, in a blinded fashion as to study timing, then reconciled scoring discrepancies by jointly reviewing the VFSS.
taVNS does not cause laryngeal or pharyngeal dysfunction in infants; rather taVNS paired with feeding improved swallowing mechanics. Measurable improvement were shown in Penetration and Aspiration Scores (PAS) in Modified Barium Swallow studies before and after 2-3 weeks of taVNS-paired feeding treatment, using a validated Modified Barium Swallow Impairment Profile.
It was also shown that there is less acute dysfunctional swallowing with taVNS turned ‘on’ or ‘off’ paired with the 3 conditions of VFSS. Specifically, PAS scores improved or did not change when taVNS was turned ‘on’ in 15/21 conditions of VFSS (71%) before starting taVNS treatment. After taVNS treatment, PAS scores improved or did not change when taVNS was turned ‘on’ in 15/17 (88%) conditions of VFSS and worsened in only 2 infants (F=6.03, p=0.026, by ANOVA). Therefore, in infants prior to starting and after a course of taVNS-paired feeding, taVNS did not result in any immediate decrement in swallowing when stimulation is turned ‘on’, and that over all patients, pharyngeal dysfunction improved with taVNS treatment by the end of treatment. This data adds considerably to evidence that delivery of taVNS is safe and positively impact swallowing in infants failing to achieve oral feeds.

This data demonstrates that infant feeding, neuroplasticity, motor skill impairment, and oropharyngeal dysphagia resulting from perinatal brain dysmaturity or injuries, including ischemic and hemorrhagic stroke, may be significantly impacted by non-invasive brain stimulation with taVNS-paired feeding.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of enhancing oromotor skills, comprising the steps of:
providing a cranial nerve stimulation system comprising at least one means of triggering activation and at least one stimulating electrode;
securing the at least one stimulating electrode adjacent to a subject’s cranial nerve;
providing the subject with a source of food;
initiating feeding upon a trigger of activation; and
administering stimulation using the at least one stimulating electrode to the cranial nerve in response to the trigger of activation.

2. The method of claim 1, wherein the at least one means of triggering activation is selected from the group consisting of: a sensing electrode noninvasively secured adjacent to a subject’s cheek or jaw muscle, a sensing mechanism integrated into a nipple or bottle used in feeding, or a manual switch.

3. The method of claim 1, wherein the trigger of activation is selected from the group consisting of: a sensing electrode sensing muscle activation that surpasses a minimum threshold, a sensing electrode sensing some displacement of food, or a manual switch being activated.

4. The method of claim 1, wherein the cranial nerve is selected from the group consisting of: the trigeminal nerve, the facial nerve, the accessory nerve, the hypoglossal nerve, the auricular branch of the vagus nerve, and the main bundle of the vagus nerve.

5. The method of claim 1, wherein the measuring step and the administering step are repeated in a closed loop.

6. The method of claim 1, wherein the administering step is performed manually in an open loop.

7. The method of claim 1, wherein the at least one stimulating electrode is noninvasively secured to a subject’s ear canal, tragus, cymba conchae, lobe, helix, anti-helix, mastoid, or neck.

8. The method of claim 1, wherein the minimum threshold is an absolute value selected from the group consisting of about: 0.1 µV, 0.5 µV, 1 µV, 5 µV, 10 µV, 50 µV, 100 µV, 200 µV, 300 µV, 400 µV, 500 µV, 1 mV, 5 mV, 10 mV, 20 mV, 30 mV, 40 mV, or 50 mV.

9. The method of claim 1, wherein the minimum threshold is a change from a base measurement taken at rest selected from the group consisting of about: 0.1 µV, 0.5 µV, 1 µV, 5 µV, 10 µV, 50 µV, 100 µV, 200 µV, 300 µV, 400 µV, 500 µV, 1 mV, 5 mV, 10 mV, 20 mV, 30 mV, 40 mV, or 50 mV.

10. The method of claim 1, wherein the minimum threshold is a percentage of a maximum potential of the muscle selected from the group consisting of about: 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95%.

11. The method of claim 1, wherein the stimulation has an intensity selected from the group consisting of about: 0.01 mA, 0.05 mA, 0.1 mA, 0.2 mA, 0.3 mA, 0.4 mA, 0.5 mA, 0.6 mA, 0.7 mA, 0.8 mA, 0.9 mA, 1 mA, 1.5 mA, 2 mA, 2.5 mA, 3 mA, 3.5 mA, 4 mA. 4.5 mA, 5 mA. 6 mA, 7 mA, 8 mA, 9 mA, and 10 mA.

12. The method of claim 1, wherein the stimulation has a frequency selected from the group consisting of about: 1 Hz, 2 Hz, 3 Hz, 4 Hz, 5 Hz, 6 Hz, 7 Hz, 8 Hz, 9 Hz, 10 Hz, 15 Hz, 20 Hz, 25 Hz, 30 Hz, 35 Hz, 40 Hz, 45 Hz, and 50 Hz.

13. The method of claim 1, wherein the stimulation has a pulse width selected from the group consisting of about: 10 µs, 20 µs, 30 µs, 40 µs, 50 µs, 60 µs, 70 µs, 80 µs, 90 µs, 100 µs, 150 µs, 200 µs, 250 µs, 300 µs, 350 µs, 400 µs, 450 µs, 500 µs, 550 µs, 600 µs, 650 µs, 700 µs, 750 µs, 800 µs, 850 µs, 900 µs, 950 µs, and 1 ms.

14. The method of claim 1, wherein the stimulation has an on duration and an off duration, each selected from the group consisting of about: 0.1 seconds, 0.5 seconds, 1.5 seconds, 2 seconds, 2.5 seconds, 3 seconds, 3.5 seconds, 4 seconds, 4.5 seconds, 5 seconds, 10 seconds, 20 seconds, 30 seconds, 40 seconds, 50 seconds, 1 minute, 2 minutes, 3 minutes, 4 minutes, 5 minutes, 10 minutes, 15 minutes, 20 minutes, 30 minutes, 40 minutes, 45 minutes, 50 minutes, and 1 hour.

15. The method of claim 1, wherein the method increases white matter neuroplasticity as measured by increased tissue anisotropy.

16. The method of claim 15, wherein tissue anisotropy is increased in white matter tracts selected from the group consisting of: the anterior limb of internal capsule, inferior front-occipital fasciculus, external capsule, and superior longitudinal fasciculus.

17. A cranial nerve stimulation system, comprising:
at least one stimulating electrode configured to attach adjacent to a cranial nerve;
at least one switch electrically connected to the at least one stimulating electrode; and
at least one stimulating unit electronically connected to the at least one stimulating electrode and the at least one switch;
wherein the at least one switch is configured to activate the at least one stimulating electrode to stimulate the cranial nerve, and wherein the at least one stimulating unit is configured to modulate at least one stimulation parameter.

18. The system of claim 17, wherein the at least one cranial nerve is selected from the group consisting of: the trigeminal nerve, the facial nerve, the accessory nerve, the hypoglossal nerve, the auricular branch of the vagus nerve, and the main bundle of the vagus nerve.

19. The system of claim 17, further comprising an impedance sensor indicating to the user whether the at least one stimulating unit has good, fair or poor contact with the patient.

20. The system of claim 17, further comprising a sensing electrode configured to attach adjacent to at least one muscle, power source, a transmitter, and a processor communicatively connected to a non-transitory computer-readable memory with instructions store thereon, which when executed by the processor, initiates a closed-loop synchronization between activation and deactivation of the at least one stimulating electrode when the at least one sensing electrode measures electrical energy in the at least one muscle that passes a minimum threshold.

21. The system of claim 17, further comprising a feeding bottle comprising at least one sensor, a power source, and a transmitter.

22. The system of claim 21, wherein the at least one sensor is selected from the group consisting of: a flow sensor, a pressure sensor, a suction sensor, a gyroscope, an accelerometer, a temperature sensor, and a volume sensor.

23. The system of claim 17, wherein the stimulating electrode comprises an earpiece and a conductive element.

24. The system of claim 21, wherein the earpiece comprises at least one outer surface conductive region positioned to directly contact a region of an ear.

25. The system of claim 21, wherein the earpiece is an anode and the conductive element is a cathode.

26. A method of enhancing muscle rehabilitation, comprising the steps of:
providing a cranial nerve stimulation system at least one stimulating electrode, at least one switch electrically connected to the at least one stimulating electrode, and at least one stimulating unit electronically connected to the at least one stimulating electrode and the at least one switch;
securing the at least one stimulating electrode to a subject’s cranial nerve;
setting at least one stimulating parameter using the at least one stimulating electrode; and
administering stimulation using the at least one stimulating electrode to the cranial nerve.

27. The method of claim 26, wherein the cranial nerve is selected from the group consisting of: the trigeminal nerve, the facial nerve, the accessory nerve, the hypoglossal nerve, the auricular branch of the vagus nerve, and the main bundle of the vagus nerve.

28. The method of claim 26, wherein the administering step is performed manually in an open loop by activating the at least one switch.

29. The method of claim 26, wherein the method further comprises a step of indicating to the user whether the at least one stimulating unit has good, fair or poor contact with the patient.

30. The method of claim 26, wherein the system further comprises at least one sensing electrode and the method further comprises a step of securing the at least one sensing electrode adjacent to a subject’s muscle group of interest, a step of measuring muscle group activation using the at least one sensing electrode that surpasses a minimum threshold, and the measuring step and the administering step are repeated in a closed loop in response to the measurement of muscle group activation surpassing the minimum threshold.
